(19) United States
(12) Patent Application Publication
(10) Pub. No.: US 2024/0050735 A1
(43) Pub. Date: Feb. 15, 2024

(54) SYSTEMS AND METHODS FOR TREATING AND INHIBITING WOUND INFECTIONS

(71) Applicant: Board of Regents of the University of Nebraska, Lincoln, NE (US)

(72) Inventors: Siwei Zhao, Omaha, NE (US); Fan Zhao, Shanghai (CN)

(73) Assignee: Board of Regents of the University of Nebraska, Lincoln, NE (US)

(21) Appl. No.: 18/493,925

(22) Filed: Oct. 25, 2023

Related U.S. Application Data
(63) Continuation-in-part of application No. 18/006,116, filed on Jan. 19, 2023.
(60) Provisional application No. 63/419,778, filed on Oct. 27, 2022.
(60) Provisional application No. 63/059,490, filed on Jul. 31, 2020.

(51) Int. Cl. A61N 1/04; A61K 31/35; A61K 38/12

(52) U.S. Cl. CPC A61N 1/0468; A61K 31/35; A61K 38/12; A61N 1/0472

(57) ABSTRACT

Systems and methods for treating and inhibiting wound infections employ a hydrogel ionic circuit (HIC)-based device for therapeutic iontophoresis and/or biofilm debridement. The HIC-based device includes: a first chamber containing a salt solution; a second chamber containing a therapeutic or buffer solution, the second chamber being configured to interface with a surface overlaying a target region; a hydrogel membrane separating the first chamber from the second chamber; and an electrode configured to apply an electrical current to the first chamber of the working device to induce an ion current in the salt solution, wherein the ion current acts on the second chamber to iontophoretically transport therapeutic molecules across the surface overlaying the target region and/or debride biofilm at the surface overlaying the target region.

### CROSS-REFERENCE TO RELATED APPLICATIONS

The present application claims priority under 35 U.S.C. § 119(e) to U.S. Provisional Application No. 63/419,778, filed Oct. 27, 2022, and titled “Systems and Methods for the Treatment of Wound Infections,” which is incorporated herein by reference in its entirety. The present application is also a Continuation-in-Part of U.S. Nonprovisional application Ser. No. 18/006,116, filed Jan. 19, 2023, and titled “HYDROGEL IONIC CIRCUIT BASED DEVICES FOR ELECTRICAL STIMULATION AND DRUG THERAPY,” which is a U.S. National Stage Application under 35 U.S.C. § 371 of International Application No. PCT/US2021/043718, filed Jul. 29, 2021, and titled “HYDROGEL IONIC CIRCUIT BASED DEVICES FOR ELECTRICAL STIMULATION AND DRUG THERAPY,” which claims priority under 35 U.S.C. § 119(e) to U.S. Provisional Application No. 63/059,490, filed Jul. 31, 2020 and titled “Hydrogel Ionic Circuit for Electrical Stimulation and Drug Therapy,” all of which are fully incorporated herein by reference.

### STATEMENT OF GOVERNMENT SUPPORT

This invention was made with U.S. government support under grant numbers U54 GM115458, P30 GM127200, R21 AR078439, and R21 AR080906 awarded by the National Institutes of Health (NIH). The U.S. government has certain rights in the invention.

### TECHNICAL FIELD

The present disclosure relates to systems and methods that employ an electrical current for therapeutic applications.

### BACKGROUND

Chronic, non-healing wounds are currently affecting 1% of the world's total population. Chronic wounds can significantly affect patients' quality of life and lead to a high rate of lower-extremity amputation. The current clinical care for chronic wounds imposes a huge burden on both patients and healthcare systems. One of the key contributing factors that lead to chronic wounds is bacterial biofilm infection, which exists in 78.2% of all chronic wounds. These biofilms arrest wounds in a prolonged inflammatory phase and inhibit skin tissue regeneration. Due to the devastating effects of biofilm, reducing bacterial bioburden is considered a critical component in chronic wound care. The latest consensus considers a bacterial count of 10⁵ colony-forming units (CFU) per gram wound tissue a critical threshold for clinical infection and healing inhibition. If the bacterial bioburden is below this threshold, a wound can generally heal in otherwise healthy patients. Above this threshold, the infection overwhelms the host immune system and stalls the wound healing process.

The current clinical standard of care for chronic wound biofilm infections includes debridement and antimicrobial treatment. Debridement physically reduces biofilms on chronic wounds. However, bacterial burden can quickly be restored within 48 hours after debridement. As a result, repeated debridement followed by long-term treatment with antimicrobial agents is currently practiced in clinics. Besides limited efficacy, conventional debridement methods can cause several safety-related issues, including dispersing bacteria to deeper tissues, overaggressive resection of healthy tissues, and pain to patients. Topical antibiotics are commonly administered for clinical wound care. However, its efficacy against chronic wound biofilm infections is found to be very limited. One reason is the high tolerance of biofilm bacteria to antibiotics. The antibiotic concentration required to kill biofilm bacteria can be 10 to 1,000 times higher than the concentration required to inhibit planktonic bacterial growth. The second reason is that bacterial communities in chronic wound biofilms are encapsulated in a matrix of protective extracellular polymeric substances (EPS), which presents a high resistance to antibiotic penetration. Multiple studies have demonstrated the decreased diffusion rate of antibiotics in various biofilms compared to their free aqueous diffusion (R. Singh, P. Ray, A. Das, M. Sharma, J Antimicrob Chemother 2010, 65, 1955; D. L. Nabb, S. Song, K. E. Kluthe, T. A. Daubert, B. E. Luedtke, A. S. Nuxoll, Front Microbiol 2019, 10, 2803; P. S. Stewart, W. M. Davison, J. N. Steenbergen, Antimicrob Agents Chemother 2009, 53, 3505; M. C. Walters, 3rd, F. Roe, A. Bugnicourt, M. J. Franklin, P. S. Stewart, Antimicrob Agents Chemother 2003, 47, 317). A 2012 study showed the diffusion coefficient of vancomycin in S. aureus biofilms was as low as 0.2 μm 2 s⁻¹, which was more than 1,000 times slower than its diffusion in water (S. Daddi Oubekka, R. Briandet, M. P. Fontaine-Aupart, K. Steenkeste, Antimicrob Agents Chemother 2012, 56, 3349).

In vivo chronic wound biofilms can be very thick. The thickness of mature P. aeruginosa biofilms can reach 200 pm above the wound surface in a rat model (R. R. Sarker, Y. Tsunoi, Y. Haruyama, S. Sato, I. Nishidate, J Biomed Opt 2022, 27, hereinafter “Sarker et al.”). S. aureus biofilms of 150 μm thick have been observed on the wound surface in mouse and pig models (H. Yang, H. Zhang, J. Wang, J. Yu, H. Wei, Sci Rep 2017, 7, 40182; S. Roy, S. Santra, A. Das, S. Dixith, M. Sinha, S. Ghatak, N. Ghosh, P. Banerjee, S. Khanna, S. Mathew-Steiner, P. D. Ghatak, B. N. Blackstone, H. M. Powell, V. K. Bergdall, D. J. Wozniak, C. K. Sen, Ann Surg 2020, 271, 1174). In vivo biofilms can also infiltrate the underlying wound tissues. Sarker et al. reported that P. aeruginosa biofilms penetrated up to 1,400 μm below the surface of rat burn wounds. S. aureus biofilm was reported to penetrate up to 190 μm below the surface of an ex vivo human skin wound infection model. Low antibiotic diffusion rates combined with high biofilm thicknesses lead to a long antibiotic diffusion time required to penetrate the entire thickness of biofilm-infected wound tissues. For example, 80.3 hours is required for vancomycin to diffuse through a S. aureus biofilm that has a thickness of 150 μm above the wound surface and infiltrates 190 μm below wound surface. As a result of such a slow antibiotic penetration, some biofilm bacteria (e.g., those in deeper layers) may be exposed to a sub-lethal concentration of antibiotics for a long period of time, which gives bacteria a chance to develop antibiotic tolerance.

Several new technologies have been explored to enhance antibiotic delivery into biofilm-infected wound tissues, but they all have their limitations. Although microneedle array can physically penetrate the EPS and reduce the diffusion distance of antibiotics, it does not increase the antibiotic diffusion speed. As a result, long-term continuous application of microneedle array on wounds (24 to 48 hours) is required to achieve a good biofilm treatment efficacy. Both pharmacological and physical disruptions of biofilm can improve antibiotic penetration in biofilms. However, the specificity of pharmacological biofilm-disruption compounds and the heterogeneity of clinical biofilms limit the applicability of these compounds. Physical disruption (e.g., laser and ultrasound), on the other hand, can cause dissemination of biofilm bacteria and damage of host tissues.

Due to the limited efficacy of the current care of chronic wound biofilm infections, a next-generation biofilm treatment strategy is critically needed.

Electric current as a biofilm treatment modality has attracted a lot of interest due to its easy application, non-invasiveness and easy dose control. Low-intensity direct current (DC) has been shown to enhance the anti-biofilm efficacy of antibiotics, which is termed the “bioelectric effect.” Some researchers have applied a 2 mA current (0.17 mA cm⁻²) to S. aureus biofilms for 24 hours in the presence of 32 μg mL⁻¹ vancomycin (J. L. del Pozo, M. S. Rouse, J. N. Mandrekar, M. F. Sampedro, J. M. Steckelberg, R. Patel, Antimicrob Agents Chemother 2009, 53, 35). This treatment resulted in a biofilm bacterial density reduction of 1 log₁₀ scale, while no antibacterial effect was observed when using vancomycin alone. A similar bioelectric effect was observed when treating in vitro S. gordonii biofilms with a 0.4 mA cm⁻² current in the presence of 2 μg mL⁻¹ gentamicin for 24 hours. After the treatment, the biofilm bacterial count was reduced by 4.3 log₁₀ scales. When an antibiotic was used alone, the bacteria count was only reduced by 0.8 log₁₀ scale. Although the working mechanism of bioelectric effect is still under investigation, enhanced antibiotic delivery by iontophoresis has been proposed as a key contributing factor. Several studies have demonstrated that iontophoresis could increase the permeation rate of antibiotics in skin tissues (S. Kaweski, R. C. Baldwin, R. K. Wong, E. K. Manders, Plastic and reconstructive surgery 1993, 92, 1342; S. Nicoli, P. Santi, Journal of controlled release 2006, 111, 89; D. Datta, D. S. Panchal, V. V. K. Venuganti, International Journal of Pharmaceutics 2021, 602, 120663). Some researchers have evaluated the transdermal iontophoresis of vancomycin (D. Datta, D. S. Panchal, V. V. K. Venuganti, International Journal of Pharmaceutics 2021, 602, 120663). Their results showed that the amount of vancomycin delivered into the epidermis by iontophoresis (0.3 mA cm⁻² current applied for 4 hours) was 2.1 times higher than that delivered by passive diffusion. In a 2006 study, it was demonstrated that transdermal iontophoresis (0.5 mA cm⁻² current applied for 2 hours) was able to increase the amount of amikacin delivered into the epidermis by 3 times compared to the amount delivered by passive diffusion (S. Nicoli, P. Santi, J Control Release 2006, 111, 89).

Although the bioelectric effect is able to enhance the efficacy of antibiotics and reduce biofilm bacterial densities, a very long treatment duration (≥24 hours) is required due to the low current intensities (≤0.5 mA cm⁻²) used. Such a long treatment duration is not practical for clinical use and will negatively affect patients' compliance. Increasing current intensity leads to a higher iontophoretic antibiotic delivery efficiency. So, the treatment duration can be reduced. However, applying high-intensity currents using conventional electrical devices can induce significant pH changes and temperature increases at the device/tissue interface due to electrochemical reactions. These side effects can lead to severe tissue damage. Besides the bioelectric effect, high-intensity pulsed electrical field has been reported to have an antibacterial effect. This effect is mainly attributed to the electroporation-induced irreversible damage to bacterial cell membranes. Electroporation also enhances the permeation of antimicrobial agents into bacterial cells. However, the high electrical energy used to treat biofilms has also been reported to cause mammalian cell membrane damage, skin tissue injuries, and neuromuscular damage. Joule heating induced by high electrical energy can also result in severe thermal damage to host tissues.

To address limitations of existing bioelectronic systems, there is a need for improved devices that can employ high current intensity while maintaining acceptable levels of pH and thermal energy at the device-to-biological tissue interface to prevent pain and/or damage to biological tissues.

### SUMMARY

Systems and methods for treating and inhibiting wound infections may employ a hydrogel ionic circuit (HIC)-based device for therapeutic iontophoresis and/or biofilm debridement. In embodiments, the HIC-based device includes a chamber containing a salt solution. The chamber is at least partially bound by a hydrogel membrane that defines a barrier for the salt solution. The HIC-based device further includes an electrode configured to apply an electrical current to the chamber to induce an ion current in the salt solution, wherein the hydrogel membrane is ionically conductive and configured to transmit the ion current.

In a sense, the HIC-based device is a buffered electrode that mitigates thermal and/or pH changes at a device-to-biological tissue interface for therapeutic applications that employ an electrical current. The HIC-based device converts the electrical current to ion current that can be transmitted through the hydrogel membrane. This may allow for the use of electrical current at higher current intensity than would otherwise be possible for electrical stimulation, iontophoresis, and other therapeutic applications that employ an electrical current.

The HIC-based device further includes a second chamber containing a therapeutic or buffer solution, wherein the second chamber is configured to interface with a surface overlaying a target region (e.g., a cutaneous wound). When the electrode applies an electrical current to the first chamber of the working device (as defined hereinafter) to induce an ion current in the salt solution, the ion current acts on the second chamber to iontophoretically transport therapeutic molecules across the surface overlaying the target region and/or debride biofilm at the surface overlaying the target region.

A HIC-based electrical biofilm treatment system may include two (or more) HIC-based devices implemented as a “working device” and a “counter device,” wherein the working device is attached to at least a portion of the target region and the counter device is attached to opposite side of (or nearby) the portion of the target region interfaced with the working device. The working device may include: a first chamber containing a salt solution; a second chamber containing a therapeutic solution, the second chamber being configured to interface with a surface overlaying a target region; a hydrogel membrane separating the first chamber from the second chamber; and an electrode configured to apply an electrical current to the first chamber of the working device to induce an ion current in the salt solution, wherein the ion current acts on the second chamber to iontophoretically transport molecules from the therapeutic solution across the surface to the target region. The counter device may include: a third chamber containing a salt solution; a fourth chamber containing a buffer solution, the fourth chamber being configured to interface with a second surface on an opposite side of or near the surface overlaying the target region; a second hydrogel membrane separating the third chamber from the fourth chamber; and a counter electrode connected to the third chamber of the counter device.

The HIC-based device/system may be utilized for a method of treating infectious biofilm affecting a wound surface. For example, the method may include steps of: disposing a salt solution within a first chamber; disposing a therapeutic or buffer solution within a second chamber, wherein the first chamber and the second chamber are separated by a hydrogel membrane; interfacing the second chamber with the wound surface; and applying an electrical current to the first chamber to induce an ion current in the salt solution, wherein the ion current acts on the second chamber to iontophoretically transport therapeutic molecules across the wound surface and/or debride biofilm at the surface overlaying the target region.

The HIC-based device/system may also be utilized for a method of treating a wound surface to inhibit the formation, reformation, and/or growth of biofilm. For example, the method may include steps of: disposing a salt solution within a first chamber; disposing an antibiotic and/or antimicrobial solution within a second chamber, wherein the first chamber and the second chamber are separated by a hydrogel membrane; interfacing the second chamber with the wound surface; and applying an electrical current to the first chamber to induce an ion current in the salt solution, wherein the ion current acts on the second chamber to iontophoretically transport antibiotic and/or antimicrobial molecules across the wound surface. In some cases, this method may be applied to wounds in order to prevent the formation of biofilm or to inhibit its growth at an early stage of infection. This method can also be applied at later stages of treatment to reduce biofilm and/or inhibit its growth or reformation.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The Detailed Description is provided with reference to the accompanying Drawings. The use of the same reference numbers in different instances in the Detailed Description and the Drawings may indicate similar or identical items. The Drawings are not necessarily to scale, and any disclosed processes may be performed in an arbitrary order, unless a certain order of steps/operations is inherent or specified in the Detailed Description or in the Claims.

FIG. 1 is a schematic illustration of a hydrogel ionic circuit (HIC)-based electrical biofilm treatment system, in accordance with an example embodiment of the present disclosure.

FIG. 2A is a schematic illustration of a HIC-based working device (e.g., anode) of the HIC-based biofilm treatment system, in accordance with an example embodiment of the present disclosure.

FIG. 2B is a schematic illustration of a conventional iontophoresis device, in accordance with an example embodiment of the present disclosure.

FIG. 2C is a chart of peak temperature on a skin surface treated with 75 mA cm⁻² current for 1 hour applied by the HIC-based working device compared to a conventional device, in accordance with an example embodiment of the present disclosure.

FIG. 2D is a chart of pH in drug/buffer chambers (filled with phosphate-buffered saline (PBS)) after 75 mA cm⁻² current application for 1 hour by the HIC-based working device compared to a conventional device, in accordance with an example embodiment of the present disclosure.

FIG. 2E is a chart of pH at skin surface after 75 mA cm⁻² current application for 1 hour by the HIC-based working device compared to a conventional device, in accordance with an example embodiment of the present disclosure.

FIG. 2F is a chart showing cell viability (normalized to untreated control) of human keratinocyte (HaCaT), human primary dermal fibroblast cells (HDFa), and human monocyte (U937) after 75 mA cm⁻² current for 1 hour applied by the HIC-based working device, in accordance with an example embodiment of the present disclosure.

FIG. 3A shows representative cryo-section images of methicillin-resistant S. aureus (MRSA) biofilm-infected porcine skin wounds after electrical application at different intensities for 1 hour, wherein all tissue samples were collected and embedded immediately after treatment, in accordance with an example embodiment of the present disclosure.

FIG. 3B is a chart of biofilm thickness above wound surface measured immediately after treatment, in accordance with an example embodiment of the present disclosure.

FIG. 3C is a chart of MRSA bacterial count (login scale) in infected skin wounds immediately after treatment measured by standard plate counting assay, in accordance with an example embodiment of the present disclosure.

FIG. 3D is a chart of bactericidal efficacy immediately after treatment, wherein bactericidal efficacy was calculated as initial bacterial count divided by bacterial count after treatment, in accordance with an example embodiment of the present disclosure.

FIG. 3E shows representative photographs of bacterial culture from MRSA biofilm-infected porcine skin wound tissues immediately after treatment, wherein all tissue homogenates were diluted 10⁶ times with PBS before plating, in accordance with an example embodiment of the present disclosure.

FIG. 4A is a chart of accumulated concentration of vancomycin (VAN) collected from skin wound tissues immediately after 1 hour iontophoresis delivery at different current intensities, wherein the drug/buffer chamber of the HIC-based working device (anode) was loaded with 1 mg mL⁻¹ VAN, in accordance with an example embodiment of the present disclosure.

FIG. 4B is a chart of permeation coefficient of VAN for 1 hour iontophoretic delivery at different current intensities, in accordance with an example embodiment of the present disclosure.

FIG. 4C is a chart of accumulated concentration of VAN in biofilm-infected skin wound tissues as a function of iontophoresis time, wherein the drug/buffer chamber of the HIC-based working device (anode) was loaded with 1 mg mL⁻¹ VAN and 75 mA cm⁻² was applied, in accordance with an example embodiment of the present disclosure.

FIG. 4D is a chart of accumulated concentrations of VAN collected from biofilm-infected skin wound tissues with different loading concentrations of VAN in the drug/buffer chamber of the working device after 75 mA cm⁻² current applied for 1 hour, in accordance with an example embodiment of the present disclosure.

FIG. 4E shows an upper panel of representative fluorescent images of cryo-sectioned skin wound samples after iontophoretic delivery of a fluorescently labeled dextran with a molecular weight of 4,000 Da (FD-4) at different current intensities for 1 hour using the HIC-based biofilm treatment system (Scale bar: 200 μm), and a lower panel of three-dimensional illustrations of the fluorescent intensity distribution in the skin tissue sample (in dashed region) shown in the upper panel, wherein the Z-axis shows fluorescent intensity in random units and X- and Y-axis have are in units of μm, in accordance with an example embodiment of the present disclosure.

FIG. 5A is a chart of MRSA bacterial count (CFU g⁻¹) in wound tissues measured at 24 hours after biofilm treatment using the HIC-based biofilm treatment system, wherein treatment duration was 1 hour, the drug/buffer chamber of the working device (anode) was loaded with 1 mg mL⁻¹ VAN, different current intensities were tested. control received no treatment, and passive diffusion used 0 mA cm⁻², in accordance with an example embodiment of the present disclosure.

FIG. 5B is a chart of bactericidal efficacy at 24 hours after biofilm treatment calculated using data in FIG. 5A, in accordance with an example embodiment of the present disclosure.

FIG. 5C is a chart of MRSA bacterial count (CFU g⁻¹) in wound tissues measured at 24 hours after biofilm treatment using the HIC-based biofilm treatment system, wherein treatment duration was 1 hour, the drug/buffer chamber of the working device (anode) was loaded with different concentrations of VAN, 75 mA cm⁻² current intensity was tested, and Control received no treatment, in accordance with an example embodiment of the present disclosure.

FIG. 5D is a chart of bactericidal efficacy at 24 hours after biofilm treatment calculated using data in FIG. 5C, in accordance with an example embodiment of the present disclosure.

FIG. 5E is a chart of anti-biofilm efficacy of repeated treatment protocols, wherein Protocol #1 applied two treatments separated by 6 hours, Protocol #2 applied two treatments separated by 24 hours, each treatment applied 75 mA cm⁻² for 1 hour using the HIC-based biofilm treatment system, the drug/buffer chamber of the working device (anode) was loaded with 1 mg mL⁻¹ VAN for all treatments, MRSA bacterial count (CFU g⁻¹) in wound tissues was measured at 24 hours after the last treatment, and control received no treatment, in accordance with an example embodiment of the present disclosure.

FIG. 5F shows representative photographs of bacterial culture from wound tissues at 24 hours after treatment (for single-treatment protocols) or after the last treatment (for repeated treatment protocols), wherein all tissue homogenates were diluted 10⁴ times with PBS before plating, in accordance with an example embodiment of the present disclosure.

FIG. 6A is a chart of accumulated concentration of daptomycin (DAP) in wound tissues measured immediately after 1 hour iontophoresis at different current intensities, wherein the drug/buffer chamber of the HIC-based working device (cathode) was loaded with 5 mg mL⁻¹ DAP, in accordance with an example embodiment of the present disclosure.

FIG. 6B is a chart of permeation coefficient (Pc) of DAP as a function of applied current intensity, in accordance with an example embodiment of the present disclosure.

FIG. 6C is a chart of accumulated concentration of DAP in wound tissues measured immediately after 1 hour iontophoresis with different DAP loading concentrations, wherein the current intensity was 75 mA cm⁻², in accordance with an example embodiment of the present disclosure.

FIG. 6D is a chart of MRSA bacterial count in wound tissues measured at 24 hours after treatment with the HIC-based biofilm treatment system (75 mA cm⁻² 1-hour) using different DAP loading concentrations, in accordance with an example embodiment of the present disclosure.

FIG. 6E shows representative photographs of bacterial colony cultured from ex vivo biofilm infected skin wounds after different treatments, wherein all tissue homogenates were diluted 10⁴ times with PBS before plating, in accordance with an example embodiment of the present disclosure.

FIG. 7A shows representative photographs of mouse in five different treatment groups (0 mA cm⁻² (sham control), 19 mAcm⁻², 38 mA cm⁻², and 75 mA cm -2 1 hour treatments applied by the HIC-based working device, and 8 mA cm⁻² 1 hour treatment applied by a conventional device, wherein the photographs were taken at 4 hours and 24 hours after treatment, in accordance with an example embodiment of the present disclosure.

FIG. 7B shows representative histological sections of skin tissues that were in direct contact with the working device using three stains (hematoxylin and eosin (H&E), Masson's trichrome, and Verhoeff-Van Gieson (VVG)), wherein skin samples were collected at 24 hours after treatment, and arrows show the detachment of epidermal from the dermis layer in 8 mA cm⁻² conventional device group, in accordance with an example embodiment of the present disclosure.

FIG. 7C is a chart of skin surface pH measured immediately after treatment, in accordance with an example embodiment of the present disclosure.

FIG. 7D is a chart of thickness of epidermal layer measured from skin sections collected at 24 hours after treatment, in accordance with an example embodiment of the present disclosure.

FIG. 8A is an experimental timeline and schematic illustration of the HIC-based biofilm treatment system setup on diabetic mouse, in accordance with an example embodiment of the present disclosure.

FIG. 8B shows representative photographs of MRSA biofilm-infected wounds in different treatment groups, in accordance with an example embodiment of the present disclosure.

FIG. 8C shows representative photographs of bacterial colony cultured from skin wounds at 24 hours after different treatments, wherein all tissue homogenates were diluted 10² times with PBS before plating, in accordance with an example embodiment of the present disclosure.

FIG. 8D is a chart of MRSA bacterial count in skin wounds measured at 24 hours after different treatments, in accordance with an example embodiment of the present disclosure.

FIG. 8E is a chart of MRSA bacterial count in skin wounds measured at 7 days after treatment, in accordance with an example embodiment of the present disclosure.

FIG. 9 is a table for gradient procedure of HPLC for vancomycin chloride detection, in accordance with an example embodiment of the present disclosure.

FIG. 10A is a chart of viable bacterial counts in collection solution of the HIC-based working device immediately after electrical debridement (75 mA cm² 60 minutes) ex vivo, wherein the result was normalized by the weight of wound tissues (per gram) and compared with the bacterial count of the untreated biofilm, in accordance with an example embodiment of the present disclosure.

FIG. 10B is a chart of viable planktonic MRSA bacteria counts before and after 75 mA cm⁻² 60 minutes treatment in vitro conducted by the HIC-based biofilm treatment system, in accordance with an example embodiment of the present disclosure.

FIG. 11A shows representative cryo-section images of ex vivo biofilm infected skin wound samples, which were incubated in 37° C. incubator for 24 hours after electrical debridement, in accordance with an example embodiment of the present disclosure.

FIG. 11B is a chart of biofilm thickness at 24 hours after electrical debridement, in accordance with an example embodiment of the present disclosure.

FIG. 11C is a chart of biofilm bioburden at 24 hours after electrical debridement characterized by the standard plate counting assay, in accordance with an example embodiment of the present disclosure.

FIG. 11D is a chart of bactericidal efficacy at 24 hours after electrical debridement, in accordance with an example embodiment of the present disclosure.

FIG. 11E is a chart of accumulated concentrations of VAN in biofilm-skin wound samples as a function of the applied time, wherein 1 mg mL⁻¹ VAN was loaded in drug/buffer chamber and applied 75 mA cm⁻² current by using HIC-based device, in accordance with an example embodiment of the present disclosure.

FIG. 12A is a chart of permeability coefficient of VAN after 75 mA cm⁻² 1-hour iontophoresis using HIC-based device loaded with different concentrations of VAN, in accordance with an example embodiment of the present disclosure.

FIG. 12B is a chart of bacteria counts after 1 mg mL⁻¹, 4.5 mg mL⁻¹, 10 mg mL⁻¹, and 20 mg mL⁻¹ VAN passively treated ex vivo mature MRSA biofilm for 24 hours, in accordance with an example embodiment of the present disclosure.

FIG. 13A is a chart of bactericidal results after 1 mg mL⁻¹ VAN passive diffusion for 24 hours (PD-24 h) on the biofilm infected skin wound, in accordance with an example embodiment of the present disclosure.

FIG. 13B is a chart of bacteria counts of ex vivo skin wound biofilm at 4 days after protocol #2 treatment and before bacterial count, in accordance with an example embodiment of the present disclosure.

FIG. 14A shows LIVE/DEAD images of HaCat cells and HDFa cells after exposed to high concentration of antibiotics in vitro, in accordance with an example embodiment of the present disclosure.

FIG. 14B is a chart of cell viability calculated from LIVE/DEAD images in ImageJ software, including: VAN-HaCaT: HaCaT cell treated by 4.5 mg mL⁻¹ vancomycin for 2 days; VAN-HDFa: HDFa cells treated by 4.5 mg mL⁻¹ vancomycin for 2 days; DAP-HaCaT: HaCaT cell treated by 20 mg mL⁻¹ daptomycin for 1 day; and DAP-HDFa: HDFa cell treated by 20 mg mL⁻¹ daptomycin for 1 day, in accordance with an example embodiment of the present disclosure.

FIG. 15A is a chart of comparative time-killing kinetics of MRSA cells in stationary phase (4-day time period) after VAN treatment by CFU counting assay, in accordance with an example embodiment of the present disclosure.

FIG. 15B is a chart of comparative time-killing kinetics of MRSA cells in stationary phase (4-day time period) after DAP treatment by CFU counting assay, in accordance with an example embodiment of the present disclosure.

FIG. 16A is a chart of permeation coefficient of DAP iontophoresis (75 mA cm⁻²) with different loading concentrations, in accordance with an example embodiment of the present disclosure.

FIG. 16B is a chart of bactericidal efficacy of DAP passive diffusion (5 mg mL⁻¹) on the ex vivo biofilm infected wounds, in accordance with an example embodiment of the present disclosure.

FIG. 17A is a chart of in vivo temperature-versus-time curve on the skin surface during different current intensity applications at 19 mA cm⁻², 38 mA cm⁻², and 75 mA cm⁻2 for 60 minutes using HIC based device and 8 mA cm⁻² 60 minutes application using conventional device, respectively, in accordance with an example embodiment of the present disclosure.

FIG. 17B is a chart of minimal inhibitory concentration (MIC) of VAN to planktonic MRSA, in accordance with an example embodiment of the present disclosure.

FIG. 18 is a schematic illustration of a test setup of VAN delivery to MRSA biofilm infected porcine skin wounds ex vivo induced by the HIC-based biofilm treatment system, wherein 10 mL phosphate salt solution was filled in counter device to minimize the pH/temperature impact of the HIC-based counter device, in accordance with an example embodiment of the present disclosure.

FIG. 19A is a chromatogram showing a 100 mg L⁻¹ antibiotic standard solution sample of vancomycin hydrochloride, in accordance with an example embodiment of the present disclosure.

FIG. 19B is a chromatogram showing a 100 mg L⁻¹ antibiotic standard solution sample of daptomycin, in accordance with an example embodiment of the present disclosure.

FIG. 20A is a graphical plot of the standard calibration curve of vancomycin hydrochloride concentrations ranging from 6.25 μg mL⁻¹ to 100 μg mL⁻¹, using optimized HPLC methods, in accordance with an example embodiment of the present disclosure.

FIG. 20B is a graphical plot of the standard calibration curve of daptomycin hydrochloride concentrations ranging from 6.25 μg mL⁻¹ to 100 μg mL⁻¹, using optimized HPLC methods, in accordance with an example embodiment of the present disclosure.

FIG. 21 shows tabular and graphical characterizations of Poly (lactic-co-glycolic acid) (PLGA) and vancomycin-encapsulated nanoparticles (PLGA/Van) nanoparticles, in accordance with an example embodiment of the present disclosure.

FIG. 22 is a chart of the vancomycin release profile of PLGA/Van in MilliQ-water at 37° C., in accordance with an example embodiment of the present disclosure.

FIG. 23 is a chart of the vancomycin level in ex vivo porcine skin wound tissues after passive diffusion and iontophoretic delivery of PLGA/Van, wherein statistical differences were determined by unpaired student's t-tests among at least 3 replicates, presented by * for p<0.05, in accordance with an example embodiment of the present disclosure.

FIG. 24 is a chart of MRSA biofilm CFU at Day 2 post-inoculation in ex vivo porcine skin wound tissues after passive diffusion and iontophoretic delivery of nanoparticles, wherein statistical differences were determined by unpaired student's t-tests among at least 3 replicates, presented by * for p<0.05, ** for p<0.01, and *** for p<0.005, in accordance with an example embodiment of the present disclosure.

### DETAILED DESCRIPTION

Biofilm infection has a high prevalence in chronic wounds and can delay wound healing. Current treatments using repeated debridement and long-term antibiotic administration impose a significant burden on patients and healthcare systems. To address their limitations, this disclosure presents a highly efficacious electrical anti-biofilm system (hereinafter the “HIC-based biofilm treatment system”).

The hydrogel ionic circuit (HIC)-based system tackles biofilm infections through two mechanisms that work simultaneously and demonstrate enhanced safety compared to conventional devices. The working mechanisms of HIC-based biofilm treatment system include: (1) efficacious electrical debridement to rapidly remove the biofilm above the wound surface; and (2) fast iontophoretic delivery of high-concentration antibiotics into biofilm and underlying wound tissues to minimize the chance of bacterial cells to adapt tolerance and rapidly reduce bacterial count. The high-intensity electric current for electrical debridement and antibiotic delivery are applied using the HIC principle discussed in International Patent Application No. PCT/US2021/043718, which is incorporated herein by reference in its entirety.

In experiments, it was demonstrated that the HIC-based biofilm treatment system was able to apply up to 75 mA cm⁻² current intensity (150 times higher than the maximal safe current intensity used by conventional transdermal iontophoresis) to ex vivo skin tissues without causing significant pH and temperature changes. The efficacy of high-intensity electrical debridement and iontophoretic antibiotic delivery of the HIC-based biofilm treatment system were then characterized separately using a porcine skin-based wound model infected with methicillin-resistant S. aureus (MRSA) biofilms. By combining these two mechanisms, a 65-minute to 2-hour treatment using the HIC-based biofilm treatment system reduced the bacterial count of MRSA biofilm-infected porcine skin wounds from over 10¹⁰ CFU g⁻¹ to 10⁵ CFU g⁻¹ at 24 hours post treatment and then to 10^{3.8} CFU g⁻¹ at 4 days post treatment. 10^{3.8} CFU g⁻¹ is below the threshold for clinical wound infection and is considered not inhibitory to wound healing. Afterwards, the in vivo safety of high-intensity currents applied by the HIC-based biofilm treatment system was studied using a healthy mouse model. The highly efficacious biofilm treatment of the HIC-based biofilm treatment system was also studied in vivo using a diabetic mouse-based wound model infected with MRSA biofilms. The in vivo anti-biofilm efficacy was consistent with the ex vivo test results. Overall, by rapidly reducing biofilm bioburden to below the clinical infection threshold, the HIC-based biofilm treatment system is able to resume the normal healing process in chronic wounds.

The HIC-based biofilm treatment system is a water-stable, hydrogel-based circuit capable of conducting ion currents across or into a lesion or wound. Within the HIC-based biofilm treatment system, a working device comprising a chamber containing a high concentration salt solution and a chamber containing a therapeutic solution are separated by hydrogel matrices. Upon application of a voltage gradient, an aqueous two-phase separation (ATPS) is formed between the hydrogel and salt solutions that stabilizes salt ions flowing from the salt solution chamber and causes their diffusion into the hydrogel or surrounding aqueous medium to be minimized. Further, this ATPS allows the hydrogel to permit ion currents to pass though onto to the therapeutic solution chamber to the targeted tissue to receive electrical stimulation and delivery of the therapeutic. Further, the HIC-based biofilm treatment system contains a counter device with the target tissue placed between the counter device and working device (e.g., the opposite side of the targeted tissue, or nearby). The counter device comprises of a buffer solution chamber and another chamber containing a high concentration salt solution both separated by hydrogel matrices. The HIC-based biofilm treatment system helps minimize the pH and temperature impact on the afflicted targeted tissue while treating the wound or biofilm with various therapeutics.

Current treatments for chronic, non-healing wounds plagued with biofilms rely on repeated debridement and long-term administration of antimicrobial agents, which can impose significant burden on patients and healthcare systems. Current techniques of debridement of lesions may result in dispersing bacteria deeper into the wound, overaggressive resection of healthy tissues and pain to the patient. Although the debridement physically reduces biofilms on chronic wounds, the biofilm's thickness, cellular encapsulation, and infiltration into the wound can lead to poor diffusion of antibiotics, regeneration, and high therapeutic tolerance. With sublethal antibiotic concentrations penetrating the biofilm despite the use of greater therapeutic concentrations for longer treatment durations, new technological advances need to be considered to have a greater efficacious effect on biofilm infected wounds.

Current treatment technologies such as microneedle arrays, ultrasound, surgical procedures, lavage, and therapeutic agents remove necrotic tissues and pathogenic biofilms, however, they have their own limitations. Physical disruption of the biofilms can assist with reducing the diffusion distance of antibiotics via penetration, but it can also cause the dissemination of the biofilm bacteria and damage the healthy tissues. Whereas therapeutic agents may not holistically target the heterogenic microbial community of the biofilm.

The implementation of low-intensity direct electric current has been shown to enhance the antibiofilm efficacy of antibiotics, termed as the “bioelectric effect,” evidenced by the reduction of the bacterial density. The enhanced antibiotic delivery by iontophoresis has been proposed to increase the transdermal permeation rate of the antibiotics. Iontophoresis creates a voltage gradient across the skin to enable transdermal drug delivery. However, such applications of low current intensities require long treatment durations for penetration. Whereas, applying high-intensity currents of existing iontophoresis devices may induce significant pH changes and temperature increases as a result of electrochemical reactions, leading to severe damage to both the biological tissue of the host and the bacterial community of the biofilm.

Here, the HIC-based device described in International Patent Application No. PCT/US2021/043718 has been modified to include a working device and a counter device to provide (1) efficacious electrical debridement to rapidly remove the biofilm above the wound surface; and (2) fast iontophoresis delivery of high-concentration antibiotics into biofilm and underlying wound tissues to minimize the potential of bacterial cells to adapt tolerance and rapidly reduce bacterial count.

In embodiments, the working device is attached to at least a portion of the target region and the counter device is attached to opposite side of (or nearby) the portion of the target region interfaced with the working device. The working device may include: a first chamber containing a salt solution; a second chamber containing a therapeutic solution, the second chamber being configured to interface with a surface overlaying a target region; a hydrogel membrane separating the first chamber from the second chamber; and an electrode configured to apply an electrical current to the first chamber of the working device to induce an ion current in the salt solution, wherein the ion current acts on the second chamber to iontophoretically transport molecules from the therapeutic solution across the surface to the target region. The counter device may include: a third chamber containing a salt solution; a fourth chamber containing a buffer solution, the fourth chamber being configured to interface with a second surface on an opposite side of or near the surface overlaying the target region; a hydrogel membrane separating the third chamber from the fourth chamber; and a counter electrode connected to the third chamber of the counter device.

The electrode connected to the working device can be anode, and the electrode connected to the counter device can be a cathode, or vice versa (i.e., cathode connected to the working device and anode connected to the counter device). The electrodes may be composed of carbon or materials with similar physical properties.

The salt solution may comprise a high concentration of sodium chloride, sodium phosphate, potassium chloride, other salt solutions not listed herein or a mixture of sodium dihydrogen phosphate and disodium phosphate solutions, or other mixtures or concentrations not listed herein.

The hydrogel membrane separating the first and second chambers or the third and fourth chambers can be comprised of materials including, but not limited to, polyethylene glycol (PEG) hydrogel membrane or the like. The therapeutic chamber can contain therapeutics that may include, but are not limited to, solutions containing antibiotics, antimicrobial agents, or the like.

By engineering this biocompatible circuit design with a working device and a counter device, therapeutic drug can be rapidly entrained across the inducible ion gradient to profuse drug across or into the lesion or wound in addition to minimizing the pH and temperature impact on the host's tissue. This device facilitates high efficiency drug delivery despite the biofilm's thickness, infiltration into the host tissue and cellular encapsulation of its associated bacterial communities.

The following disclosure provides example embodiments of the HIC-based electrical biofilm treatment system and experimental findings associated with example implementations of the HIC-based electrical biofilm treatment system and/or methods/processes that use the HIC-based electrical biofilm treatment system.

Example Embodiment of the HIC-based electrical biofilm treatment system

One challenge of conventional electrical biofilm treatment devices is the significant pH change at device/tissue interface when high-intensity current is applied. Current electrical devices typically conduct electron currents. They have to be converted to ion currents at the device/tissue interface via electrochemical (EC) reactions, which require the decomposition of water molecules. Water electrolysis generates hydrogen ions at the anode and hydroxide ions at the cathode. When high-intensity currents are applied, these ions cannot be sufficiently buffered by conventional electrical devices due to the limited pH buffering capacity of their buffering systems (e.g., phosphate-buffered saline (PBS), which contains only 12 mM phosphate ions). The accumulated acidic and alkaline concentrations result in chemical burns of skin tissues. Non-polarizable electrodes (e.g., silver/silver chloride) are capable of transferring charges without splitting water molecules. However, if the current intensity exceeds their charge transfer capacity, water decomposition can still be triggered to cause significant pH changes. Another problem of conventional electrical devices is the thermal effect during high-intensity current application. Water molecule decomposition requires 1.48 V DC potential, but the voltage across the electrode/media interface is usually higher than 1.48 V and it increases with increasing current intensity. The excess voltage (i.e., electrode over-potential) can result in temperature increase on tissue surface. In addition, the Joule heating generated by electric current conduction can also contribute to thermal damage to skin tissues.

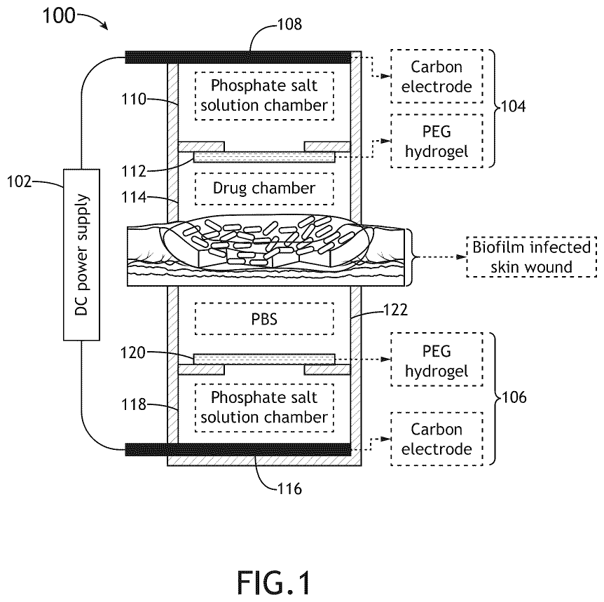

To overcome problems induced by conventional electrical devices when applying high-intensity currents, a hydrogel ionic circuit (HIC)-based biofilm treatment system 100 for combating bacterial infection has been developed. An example embodiment of the HIC-based electrical biofilm treatment system 100 is illustrated in FIG. 1.

The HIC-based biofilm treatment system 100 includes a working device 104 configured to be interfaced with (e.g., attached and/or placed into contact with) a surface overlaying a target region. The target region may include, but is not limited to, a cutaneous or subcutaneous wound plagued with or without an infectious biofilm. For example, the working device may be placed into contact with the surface of a wound or biofilm-infected wound. The HIC-based biofilm treatment system 100 further includes a counter device 106 configured to be interfaced with a surface that is on an opposite side of or near the surface overlaying the target region. For example, the counter device 106 may be placed into contact with the surface of skin/tissue on the opposite side of, or skin/tissue that is adjacent to, the surface of the biofilm-infected wound. The working device 104 and the counter device 106 may both comprise HIC-based devices, and either device can be configured as the circuit's anode or cathode. In some embodiments, whether the working device 104 is configured as the anode or the cathode will depend on the polarity of the iontophoresis required to deliver antibiotics or other therapeutic agents. The circuit formed by the working device 104 and the counter device 106 may be powered by a direct current (DC) power source 102 (e.g., a DC power supply, battery, etc.).

In embodiments, the HIC-based working device 104 includes or is coupled to an electrode 108 (e.g., a carbon/metal electrode) configured to apply electrical current to a salt solution chamber 110 containing a salt solution (e.g., a sodium chloride solution, a sodium phosphate solution, a potassium chloride solution, a sodium dihydrogen phosphate solution, a disodium phosphate solution, any mixture of the foregoing solutions, or the like). In some embodiments, the salt solution chamber 110 is filled with a high-concentration mixture of sodium dihydrogen phosphate and/or disodium phosphate solution. The electrode 108 conducts electrical current provided by the DC power supply 102. For example, the electrode 108 may be coupled to the working (positive or negative) terminal of the DC power supply 102.

The salt solution chamber 110 may include or may be coupled to a hydrogel membrane 112 (e.g., PEG hydrogel matrix) that defines a barrier for the salt solution contained in the salt solution chamber 110. The HIC-based working device 104 further includes a drug/buffer chamber 114 containing a therapeutic solution (e.g., a solution containing an antibiotic and/or antimicrobial therapeutic agent). In some embodiments, the drug/buffer chamber 114 is loaded with a buffer (e.g., PBS) instead, in order to perform biofilm debridement without iontophoretic drug delivery. Although, as later discussed herein, the combination of biofilm debridement and iontophoretic drug delivery has been shown to better combat biofilm than either one of the techniques on its own.

Chambers 110 and 114 may be coupled together or formed from separate compartments within a common structure, with the hydrogel membrane 112 being disposed between the chambers and configured to separate the salt solution in chamber 110 from the therapeutic solution (or buffer) in chamber 114. The hydrogel membrane 112 may be ionically conductive and configured to permit certain ions (e.g., Na⁺, Cl⁻) to flow between the chambers 110 and 114 while salt ions (e.g., phosphate, sulfate, citrate, carbonate, or polyacrylate salt ions) of the salt solution are stably contained in chamber 110 due to ATPS. The PEG hydrogel membrane 112 forms a unique ATPS with the high-concentration phosphate salt solution. The ATPS minimizes the diffusion of phosphate salt ions to the drug/buffer chamber 114 to avoid osmolarity changes in the drug/buffer chamber 114 and tissues.

The drug/buffer chamber 114 may be configured to interface with a surface overlaying a target region. The target region may include, but is not limited to, a cutaneous or subcutaneous wound plagued with or without an infectious biofilm. For example, the drug/buffer chamber 114 of the working device 104 may be placed into contact with the surface of a wound or biofilm-infected wound. In some embodiments, the drug/buffer chamber 114 may have an opening or permeable/semi-permeable membrane configured to be placed into contact with the surface overlaying the target region.

When the HIC-based biofilm treatment system 100 is in use, the electrode 108 is configured to conduct electrical current from the DC power source 102 to the salt solution chamber 110, wherein the electrical current induces an ion current in the salt solution. This ion current is transmitted to (or induces a second ion current within) the drug/buffer chamber 114 via the hydrogel membrane 112 while salt ions are stably contained in chamber 110 due to ATPS. The ion current acts on (e.g., is transmitted to/through) chamber 114 to iontophoretically transport molecules (e.g., antibiotic/antimicrobial molecules, such as vancomycin molecules, daptomycin molecules, etc., or any combination thereof) from the therapeutic solution across the surface overlaying the target region (e.g., across the surface of the biofilm-infected wound and into the biofilm and/or underlying tissue). In addition to, or in the absence of, iontophoretic transport of therapeutic agents across the surface overlaying the target region, the working device 104 is configured to debride biofilm with electrostatic force generated by the ion current acting on the drug/buffer chamber 114.

The HIC-based counter device 106 includes a counter electrode 116 (e.g., a carbon/metal electrode) that is connected to ground or to a counter-terminal of the DC power supply 102 so that the working device 104 and the counter device 106 provide a complete circuit. The counter electrode 116 is also connected to a salt solution chamber 118 containing a salt solution (e.g., a sodium chloride solution, a sodium phosphate solution, a potassium chloride solution, a sodium dihydrogen phosphate solution, a disodium phosphate solution, any mixture of the foregoing solutions, or the like). In some embodiments, the salt solution chamber 118 is filled with a high-concentration mixture of sodium dihydrogen phosphate and/or disodium phosphate solution.

The salt solution chamber 118 may include or may be coupled to a hydrogel membrane 120 (e.g., PEG hydrogel matrix) that defines a barrier for the salt solution contained in the salt solution chamber 118. The HIC-based counter device 106 further includes a buffer chamber 122 containing a buffer solution (e.g., PBS). Chambers 118 and 122 may be coupled together or formed from separate compartments within a common structure, with the hydrogel membrane 120 being disposed between the chambers and configured to separate the salt solution in chamber 118 from the buffer solution in chamber 122. The hydrogel membrane 120 may be ionically conductive and configured to permit certain ions (e.g., Na⁺, Cl⁻) to flow between the chambers 118 and 122 while salt ions (e.g., phosphate, sulfate, citrate, carbonate, or polyacrylate salt ions) of the salt solution are stably contained in chamber 118 due to ATPS. The PEG hydrogel membrane 120 forms a unique ATPS with the high-concentration phosphate salt solution. The ATPS minimizes the diffusion of phosphate salt ions to the buffer chamber 122 to avoid osmolarity changes in the buffer chamber 122 and tissues.

The buffer chamber 122 may be configured to interface with a surface that is on an opposite side of or near the surface overlaying the target region. For example, the buffer chamber 122 may be placed into contact with the surface of skin/tissue on the opposite side of, or skin/tissue that is adjacent to, the surface of a biofilm-infected wound. In some embodiments, the buffer chamber 122 may have an opening or permeable/semi-permeable membrane configured to be placed into contact with the surface that is on an opposite side of or near the surface overlaying the target region.

When the HIC-based biofilm treatment system 100 is in use, the working device 104 and the counter device 106 provide a complete circuit so that DC electrical current from the DC power source 102 can be conducted across the surface overlaying the target region (e.g., across the biofilm-infected wound) for iontophoretic transport of therapeutic modules and/or for the purpose of electrical biofilm debridement. However, as discussed and demonstrated in this disclosure, the HIC-based devices 104 and 106 can conduct high-intensity currents without drastic changes in temperature and pH at the interface between the HIC-based devices 104 and 106 and skin tissue. In some embodiments, the salt solution contains an amount of salt required to maintain a pH in the range of 6.5 to 8.5 at the surface overlaying the target region, and the salt solution is configured to absorb enough of the heat generated by electrode overpotential to maintain a temperature below 43° C. at the surface overlaying the target region.

During biofilm treatment, the DC power source 102 applies a DC signal to the working and counter devices 104 and 106. The hydrogen/hydroxide ions generated by EC reaction are neutralized by the HIC-based biofilm treatment system 100, enabled by the high-concentration phosphate salt ions in phosphate salt solution chambers 110 and 118 (e.g., containing 600 to 1080 mM phosphate salt ions). The EC reaction-induced heat is absorbed by the high water content in the devices. In addition, the Joule heat produced by current conduction is minimized due to the high electrical conductivity of the high-concentration phosphate salt solutions. These features allow for the application of current intensities that are significantly higher than the safe current intensity used by conventional electrical devices (e.g., 0.5 mA cm⁻² is typically applied by conventional transdermal iontophoresis) without causing tissue damage. The PEG hydrogel 112 forms a unique ATPS with the high-concentration phosphate salt solutions. The ATPS minimizes the diffusion of phosphate salt ions to the drug/buffer chamber 114 to avoid osmolarity changes in the drug/buffer chamber 114 and tissues. The ion currents are transmitted to the drug/buffer chamber 114 through the PEG hydrogel 112 and then to the biofilm-infected wound tissue. In the drug/buffer chamber 114, the high-intensity ion current mobilizes the antibiotics and iontophoretically delivers them into the biofilm and the underlying wound tissue with a high permeation rate. Furthermore, in the wound tissue, the high-intensity ion current electrically debrides the biofilm.

The HIC-based biofilm treatment system 100 may be utilized for a method of treating infectious biofilm affecting a wound surface. For example, the method may include steps of: disposing a salt solution within a first chamber (chamber 110); disposing a therapeutic or buffer solution within a second chamber (chamber 114), wherein the first chamber (chamber 110) and the second chamber (chamber 114) are separated by a hydrogel membrane (hydrogel membrane 112); interfacing the second chamber (chamber 114) with the wound surface; and applying an electrical current to the first chamber (chamber 110) to induce an ion current in the salt solution, wherein the ion current acts on the second chamber (chamber 114) to iontophoretically transport therapeutic molecules across the wound surface and/or debride biofilm at the wound surface.

The HIC-based biofilm treatment system 100 may also be utilized for a method of treating a wound surface to inhibit the formation, reformation, and/or growth of biofilm. For example, the method may include steps of: disposing a salt solution within a first chamber (chamber 110); disposing an antibiotic and/or antimicrobial solution within a second chamber (chamber 114), wherein the first chamber (chamber 110) and the second chamber (chamber 114) are separated by a hydrogel membrane (hydrogel membrane 112); interfacing the second chamber (chamber 114) with the wound surface; and applying an electrical current to the first chamber (chamber 110) to induce an ion current in the salt solution, wherein the ion current acts on the second chamber (chamber 114) to iontophoretically transport antibiotic and/or antimicrobial molecules across the wound surface. In some cases, this method may be applied to wounds in order to prevent the formation of biofilm or to inhibit its growth at an early stage of infection. This method can also be applied at later stages of treatment to reduce biofilm and/or inhibit its growth or reformation.

In vitro and ex vivo Safety Evaluation of High-Intensity Current Applied by the HIC-Based Electrical Biofilm Treatment System

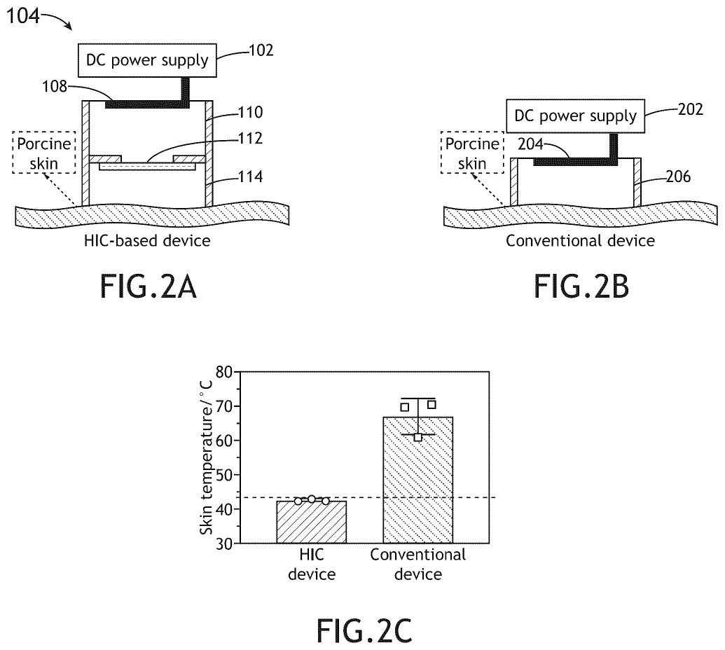

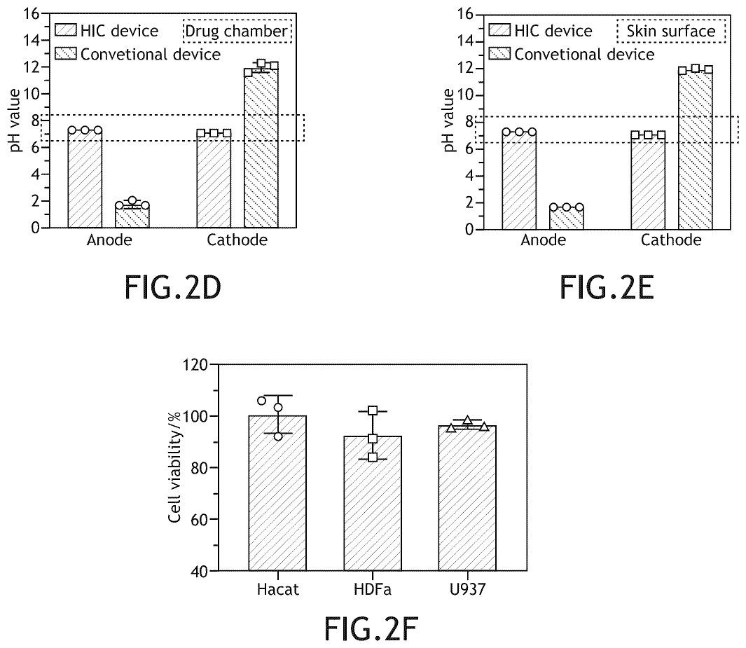

In vitro and ex vivo safety of the HIC-based biofilm treatment system 100 has been evaluated when applying a high current intensity of 75 mA cm⁻² for 1 hour. This current intensity is 150 times higher than the maximal safe current (0.5 mA cm⁻²) used by conventional transdermal iontophoresis. First, the temperature and pH changes induced by 75 mA cm⁻² current application were measured using fetal porcine skins (see FIG. 2A). In this test, the drug/buffer chamber 114 of the HIC-based device 104 was filled with PBS. FIG. 2B illustrates a conventional electrical device that was tested as a comparison. The conventional device was constructed by directly inserting a carbon electrode 204 in the PBS-filled drug/buffer chamber 206 and powering the electrode 204 using a DC power supply 202. During the 1 hour current application, the surface temperature of the skin in contact with the working device 104 (anode) was monitored.

Based on previous experience, HIC-based anode devices always generated more heat than HIC-based cathode devices, so the use of the HIC-based device 104 in an anode configuration represented the worst case. To generate less heat at the surface overlaying the target region, it may be desirable to use the HIC-based device 104 in a cathode configuration (i.e., wherein the counter device 106 is configured as the anode).

The average peak skin surface temperature treated by the HIC-based biofilm treatment system was 42.5±0.32° C., which was lower than the maximal safe temperature (43° C.) that skin can tolerate. In contrast, the conventional device increased skin surface temperature up to 67.0±5.29° C. (see FIG. 2C), which was considerably higher than the safe temperature threshold. Measurements of the pH in the drug/buffer chambers were also performed and on the surface of the skin at device contact immediately after electrical application. When HIC-based devices were used, the pH in both locations remained between 6.5 and 8.5, which is considered safe for skin tissues. The conventional device, however, changed the pH to around 2 on the anode side and around 12 on the cathode side (see FIGS. 2D and 2E). The significant pH changes were owing to the hydrogen ions and hydroxide ions generated on anode and cathode, respectively, from EC reactions. These ions cannot be sufficiently neutralized by the pH-buffering system used in the conventional device (e.g., PBS) due to its low phosphate ion concentration (12 mM). Hence, these results demonstrated the ability of the HIC-based biofilm treatment system 100 to maintain a safe pH and temperature on skin surface during high-intensity current application to avoid thermal and chemical damage to skin tissues.

Since the HIC-based biofilm treatment system 100 did not cause significant pH and temperature changes when applying high-intensity current to tissues, testing went on to evaluate the cytotoxicity of high-intensity current itself applied by the HIC-based biofilm treatment system 100. Here, the viability of in vitro cultured skin wound-related cells was tested after being exposed to 75 mA cm⁻² current applied by the HIC-based biofilm treatment system for 1 hour. Human keratinocyte (HaCaT), human primary dermal fibroblast cells (HDFa), and human monocyte (U937) were tested. The results showed that treatment with 75 mA cm⁻² current for 1 hour had minimal impact on the viability of these cells (see FIG. 2F), which provided further evidence for the safety of the HIC-based biofilm treatment system 100.

Ex vivo Electrical Debridement of Biofilm Induced by the HIC-Based Electrical Biofilm Treatment System

The high-intensity electrical biofilm debridement efficacy of the HIC-based biofilm treatment system 100 has been studied using an ex vivo MRSA biofilm-infected porcine skin wound. An excisional skin wound was created by a 6-mm biopsy punch down to the dermis layer. The wound was then inoculated with 20 μL of 1×10⁸ CFU mL⁻¹ MRSA bacteria solution. The bacteria were allowed to grow for 48 hours to form a mature biofilm. The HIC-based biofilm treatment system 100 was then used to apply current to the infected wound for 1 hour. The drug/buffer chamber 114 of the HIC-based biofilm treatment system 100 was loaded with PBS in this test. Different current intensities, including 0 mA cm⁻² (untreated control), 0.5 mA cm⁻², 19 mA cm⁻², 38 mA cm⁻², and 75 mA cm⁻², were tested.

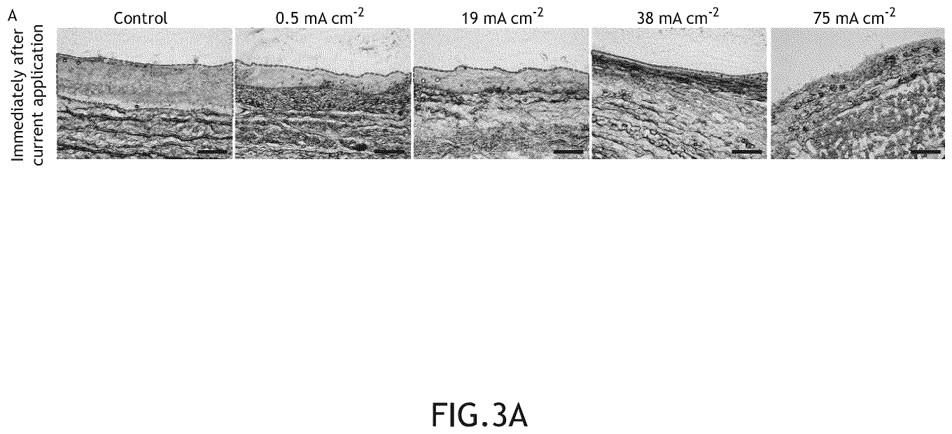

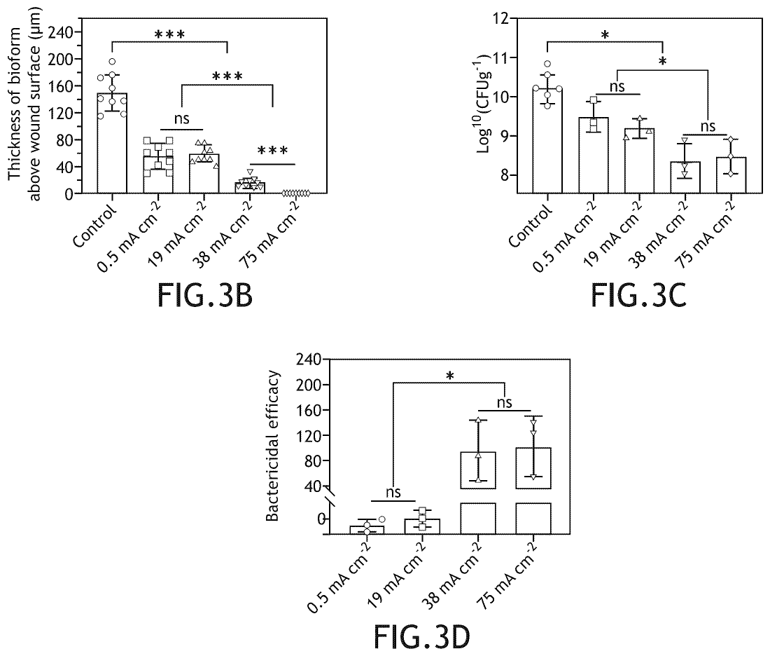

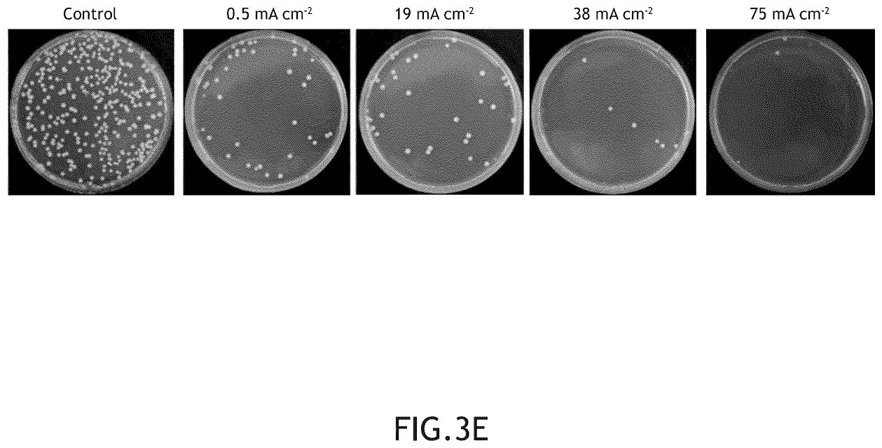

After electrical application, the biofilm structure was evaluated. As shown in FIG. 3A, when no current was applied, a thick biofilm could be seen on the surface of the wound. The thickness of this biofilm was 149.8±27.81 μm (see FIG. 3B), which was similar to previously reported thickness of in vivo chronic wound biofilms, indicating the formation of a mature biofilm. The thickness of the MRSA biofilm was reduced to 55.3±18.96 μm and 58.6±12.72 μm after low current intensity treatments at 0.5 mA cm⁻² and 19 mA cm⁻², respectively. When the current intensity further increased to 38 mA cm² and 75 mA cm⁻², more biofilm thickness reduction was observed. The biofilm thickness was reduced to 14.6±12.72 μm after 38 mA cm⁻² treatment, and no discernible biofilm could be observed on wound surface under microscope after 75 mA cm⁻² treatment. These results highlighted the structural damage to biofilm induced by high-intensity current application.

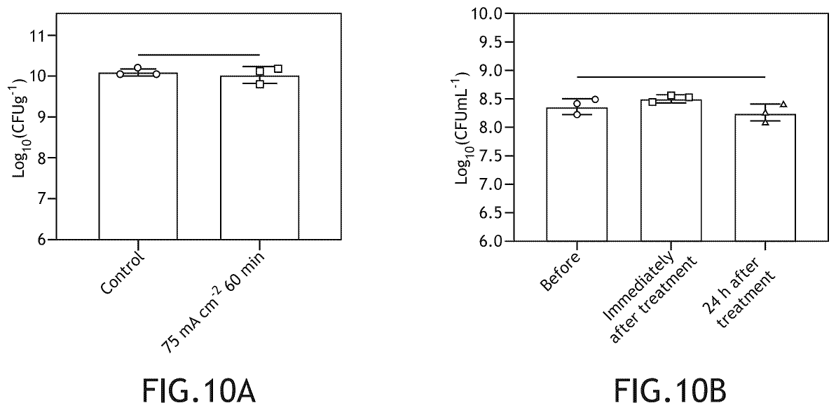

The biofilm debridement effect of low-intensity electrical current treatment has been reported previously. The main mechanism of electrical debridement was attributed to physical biofilm disruption and detachment caused by the current-induced electrostatic force that pulled biofilm bacteria away from their substrate. For example, it has been demonstrated that 15 μA cm⁻² current applied for 60 minutes induced 80% detachment of P. aeruginosa biofilm from a glass surface (S. H. Hong, J. Jeong, S. Shim, H. Kang, S. Kwon, K. H. Ahn, J. Yoon, Biotechnol Bioeng 2008, 100, 379). Another study has reported 78% detachment of S. epidermidis biofilm and 54% detachment of S. aureus biofilm from surgical stainless steel after applying 15-125 μA current for 2.5 hours (A. Van der Borden, H. Van der Mei, H. Busscher, Journal of Biomedical Materials Research Part B: Applied Biomaterials: An Official Journal of The Society for Biomaterials, The Japanese Society for Biomaterials, and The Australian Society for Biomaterials and the Korean Society for Biomaterials 2004, 68, 160). In addition to physical biofilm disruption and detachment, it has also been proposed that the adverse effects of electrochemical reactions may cause bacterial cell death. However, there is no consensus on the contribution of this effect, as some studies reported no bacterial killing induced by electric current alone (J. W. Costerton, B. Ellis, K. Lam, F. Johnson, A. E. Khoury, Antimicrobial agents and chemotherapy 1994, 38, 2803; J. Jass, H. M. Lappin-Scott, Journal of Antimicrobial Chemotherapy 1996, 38, 987; J. Jass, J. W. Costerton, H. Lappin-Scott, Journal of industrial microbiology and biotechnology 1995, 15, 234). To investigate this, the solution in the drug/buffer chamber 114 of the working device 104 was collected immediately after the high-intensity electrical debridement and measured the bacterial density. It was found that the density of viable bacteria in the collection solution was similar to the bacterial count in the untreated control biofilm (see FIG. 10A). This result suggested that the high-intensity current applied by the HIC-based biofilm treatment system 100 had no bacterial killing effect. To further verify this result, an in vitro experiment was also performed by applying 75 mA cm⁻² current to planktonic MRSA bacteria for 60 minutes using the HIC-based biofilm treatment system 100. The number of viable bacteria was counted before, immediately after and at 24 hours after treatment (see FIG. 10B). No significant difference in viable bacterial count was observed among these three time-points, further confirming the lack of bacterial killing by the high-intensity current applied by the HIC-based biofilm treatment system 100. This result was also consistent with the in vitro and ex vivo safety study which showed that the HIC-based biofilm treatment system 100 could effectively minimize pH and temperature changes induced by high-intensity current application and damage to mammalian cells.

Next, quantitative measurements of the bacterial count remaining in the wound tissue after current application were taken at different intensities using the standard spread counting assay (see FIGS. 3C and 3E). The untreated wound sample had a MRSA bacterial count of 10^{10.3} CFU g⁻¹. The bacterial count was reduced to 10^{9.3} CFU g⁻¹ and 10^{9.2} CFU g⁻¹ after low-intensity current application at 0.5 mA cm⁻² and 19 mA cm⁻², respectively. When high current intensities of 38 mA cm⁻² and 75 mA cm⁻² were used, the bacterial count was further reduced to 10^{8.4} CFU g⁻¹ and 10^{8.5} CFU g⁻¹, respectively.

The bactericidal efficacy, defined as initial bacterial count divided by remaining bacterial count after treatment, was 5.9±4.05, 10.2±5.2, 96.6±47.84 and 102.7±48.82 times for 0.5 mA cm⁻², 19 mA cm⁻², 38 mA cm⁻² and 75 mA cm⁻² treatment, respectively (see FIG. 3D). The bactericidal efficacy at 38 mA cm⁻² and 75 mA cm⁻² was equivalent to a 99% bactericidal rate. The biofilm thickness and bacterial count measurements showed that 75 mA cm⁻² applied for 1 hour by the HIC-based biofilm treatment system effectively destroyed mature MRSA biofilm above wound surface and removed a majority (>99%) of the bacteria.

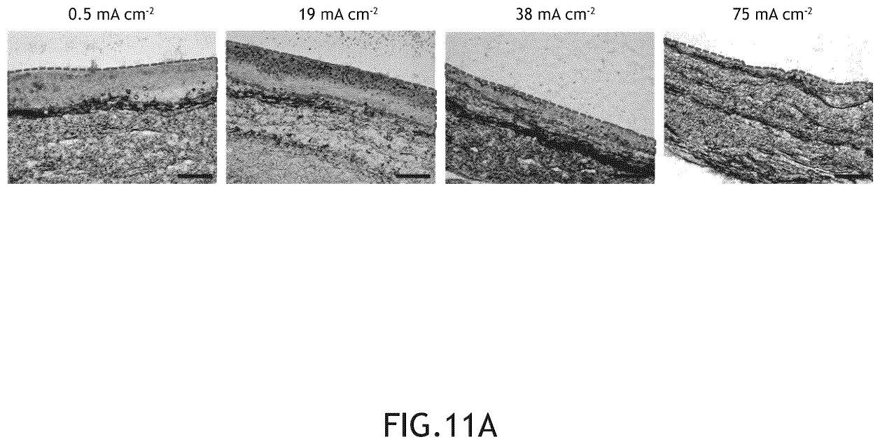

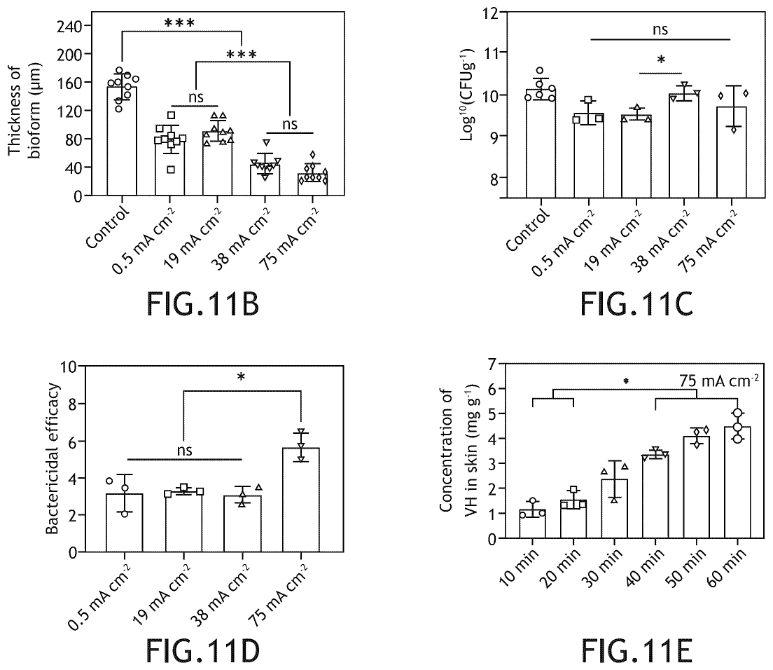

Although the HIC-based biofilm treatment system 100 achieved a biofilm debridement efficacy that was better than the conventional low current intensity method, the HIC-based biofilm treatment system 100 did not prevent biofilm reformation similar to conventional debridement methods (see FIG. 11A). The biofilm thickness recovered to 79.0±20.54 μm and 91.1±14.66 μm at 24 hours after electrical debridement at 0.5 mA cm⁻² and 19 mA cm⁻², respectively (see FIG. 11B). The restoration of biofilm was also observed in high current intensity treatment groups. The biofilm thickness was recovered to 43.4±13.19 μm and 28.9±7.99 μm at 24 hours after electrical debridement at 38 mA cm⁻² and 75 mA cm⁻², respectively. The restoration of biofilm was further evidenced by bacterial count, which was above 10^{9.1} CFU g⁻¹ in all groups at 24 hours after electrical debridement (see FIG. 11C). Accordingly, the bactericidal efficacy was decreased (see FIG. 11D) compared to that immediately after electrical debridement. These results suggested that although high-intensity currents alone were able to quickly debride mature biofilm, it did not prevent biofilm recovery after treatment.

### High-Intensity Iontophoretic Delivery of Vancomycin (VAN)

Investigations were also performed to determine the iontophoretic antibiotic delivery efficiency of the HIC-based biofilm treatment system 100 using high current intensities. These investigations included an ex vivo antibiotic delivery study using the same MRSA biofilm-infected porcine skin wound model as in the previous section. Vancomycin (VAN) was used here as a model drug, since it is the FDA-approved antibiotic and is the clinical gold standard for combating S. aureus infections.

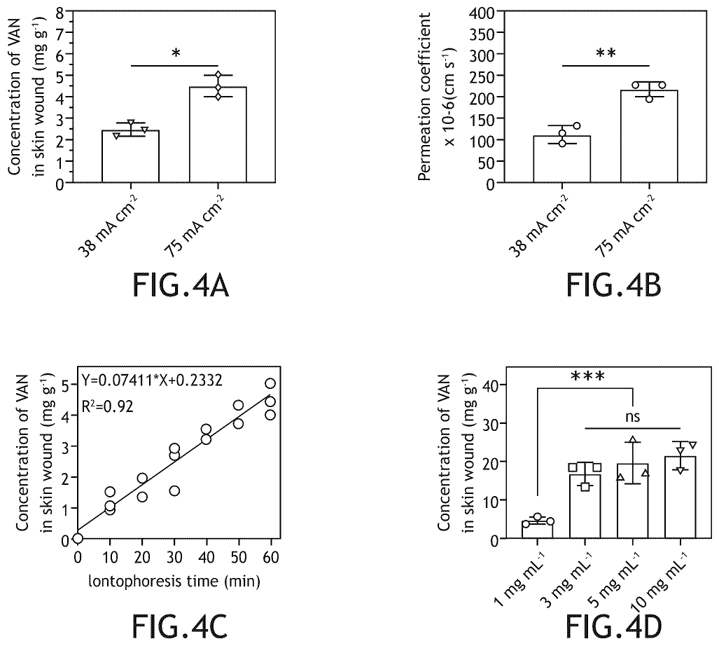

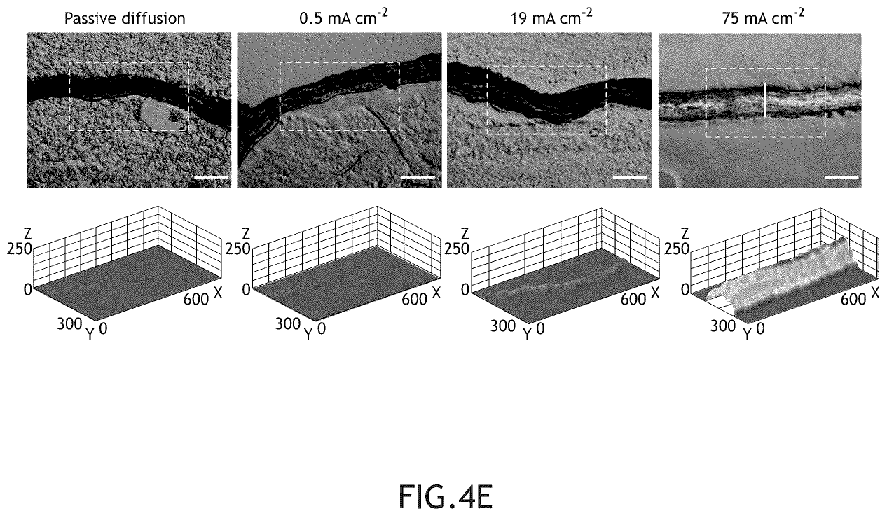

First the VAN delivery efficiency was tested at different current intensities (from 0 mA cm⁻² (passive diffusion) to 75 mA cm⁻²) applied by the HIC-based biofilm treatment system 100 for 1 hour. The drug/buffer chamber 114 of the working device 104 was loaded with 1 mg mL⁻¹ VAN in this study. The anode side was used as the working device 104 because VAN required anodal iontophoresis. The accumulated concentration of VAN in biofilm infected skin wound was measured immediately after iontophoresis using high performance liquid chromatography (HPLC). As shown in FIG. 4A, when 38 mA cm⁻² was used, the accumulated concentration of VAN in wound tissue reached 2.5±0.32 mg g⁻¹. When 75 mA cm⁻² was used, the VAN concentration in wound tissue increased to 4.5±0.52 mg g⁻¹. The VAN concentration in wound tissue after passive diffusion, 0.5 mA cm⁻², and 19 mA cm⁻² iontophoresis were also measured. However, they could not be distinguished from the background interference of the residual biofilm components, so no quantitative results were obtained (data not shown). Importantly, the VAN concentration delivered by 75 mA cm⁻² iontophoresis for 1 hour exceeded the minimal biofilm eradication concentration (MBEC) of VAN reported for MRSA biofilm, which was 4 mg g⁻¹. The ability to deliver a lethal antibiotic concentration into biofilm-infected wounds within a short period of time was critical for biofilm treatment as it would minimize the chance for bacteria to develop adaptive tolerance.

The VAN permeation coefficient (Pc) achieved by 38 mA cm⁻² and 75 mA cm⁻² iontophoresis were calculated (see FIG. 4B). At 38 mA cm⁻², the Pc was 112.0±20.97×10⁻⁶ cm s⁻¹. It increased to 216.5±18.96×10⁻⁶ cm s⁻¹ when 75 mA cm⁻² was used. This was a significant improvement from conventional low-intensity transdermal iontophoresis. It has previously been reported that a conventional transdermal iontophoresis using 0.31 mA cm⁻² can achieve a VAN Pc of 0.221×10⁶ cm s⁻¹ in excised porcine skin tissues. This was 980 times lower than the VAN Pc achieved by 75 mA cm⁻² iontophoresis using the HIC-based biofilm treatment system 100. This substantial increase in VAN Pc achieved by the HIC-based biofilm treatment system 100 was attributed to the higher current intensity used and the enhanced wound tissue permeability induced by the moderate wound temperature increase from electric current application.

Next, the progression of VAN accumulation in wound tissues was evaluated at different time points during the 75 mA cm⁻² 1 hour iontophoresis application (see FIG. 4C and FIG. 11E). The drug/buffer chamber of the working device was loaded with 1 mg mL⁻¹ VAN in this test. The results showed that VAN concentration increased almost linearly with time (R²=0.92).

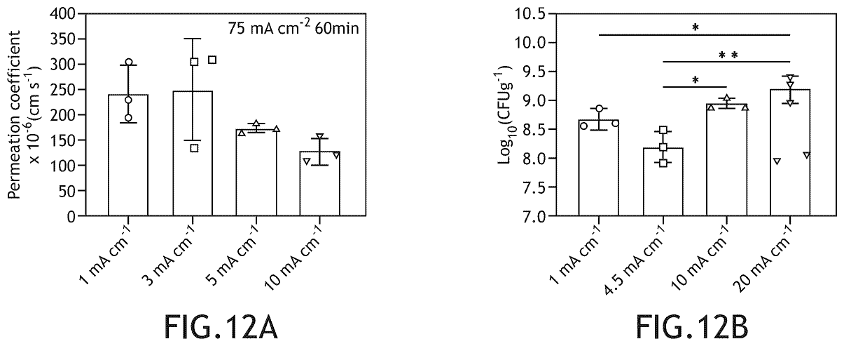

Since iontophoretic drug delivery efficiency was highly dependent on drug loading concentration, testing went on to determine the effect of VAN loading concentration on its iontophoretic delivery efficacy (using 75 mA cm⁻² 1 hour) by loading the drug/buffer chamber with 1 mg mL⁻¹, 3 mg mL⁻¹, 5 mg mL⁻¹, and 10 mg mL⁻¹ VAN. The results showed that the VAN concentration in wound tissue was significantly increased to 16.7±2.96 mg g⁻¹ when 3 mg mL⁻¹ VAN was used (see FIG. 4D). However, there was no further increase in VAN tissue concentration that was statistically significant when higher VAN loading concentrations (5 mg mL⁻¹, and 10 mg mL⁻¹) were used. As a result, the calculated Pc for 5 mg mL⁻¹, and 10 mg mL⁻¹ loading concentrations was decreased compared to the Pc achieved by 1 mg mL⁻¹, and 3 mg mL⁻¹ loading concentrations (see FIG. 12A).

It was determined that high-intensity iontophoresis using the HIC-based biofilm treatment system 100 was able to enhance the VAN penetration depth in wound tissues. A high penetration depth would allow effective treatment of bacteria in deeper layers of clinical biofilm infections to minimize biofilm reformation. A fluorescently labeled dextran (M.W.=4 kDa, FD-4) was used to visualize drug distribution in wound tissue immediately after iontophoresis in this test. The hydrodynamic radius and net charge of FD-4 were similar to VAN, so FD-4 was expected to exhibit a similar iontophoretic behavior as VAN. The upper panel of FIG. 4E shows the distribution of FD-4 in wound tissue cross-sections after passive diffusion, 0.5 mA cm⁻², 19 mA cm⁻², and 75 mA cm⁻² iontophoresis applied by the HIC-based biofilm treatment system 100 (loaded with 1 mg mL⁻¹ FD-4 in drug/buffer chamber) for 1 hour. All wound tissues were dehydrated by acetone before sectioning to minimize FD-4 mobility during sample processing. When passive diffusion and 0.5 mA cm⁻² were applied, the fluorescence was indiscernible under microscope. When 19 mA cm⁻² was applied, fluorescence could only be seen near the upper boundary of the wound tissue facing the working device during iontophoresis. In contrast, after 75 mA cm⁻² iontophoresis for 1 hour, FD-4 penetrated the entire thickness of the tissue sample, which was 955.7±192.56 μm thick before dehydration and 182.4±12.62 μm thick after dehydration. This penetration depth was higher than the typical thickness of in vivo S. aureus biofilm infections in skin wounds (150 μm above wound surface and 190 μm below wound surface). The lower panel of FIG. 4E shows the distribution of FD-4 reconstructed in a three-dimensional format with the Z-axis showing the fluorescent intensity. The results in this section provided both quantitative and qualitative evidence to show that high-intensity iontophoresis applied by the HIC-based biofilm treatment system 100 not only increased VAN permeation rate, but also enhanced VAN penetration depth in wound tissues.

### The Combined Anti-Biofilm Efficacy of High-Intensity Electrical Debridement and Iontophoretic VAN Delivery

The previous sections demonstrate that high-intensity current application physically removes biofilm biomass and high-intensity iontophoresis significantly enhances VAN delivery into wound tissues. This section discusses the anti-biofilm efficacy of the HIC-based biofilm treatment system 100 when combining these two effects.

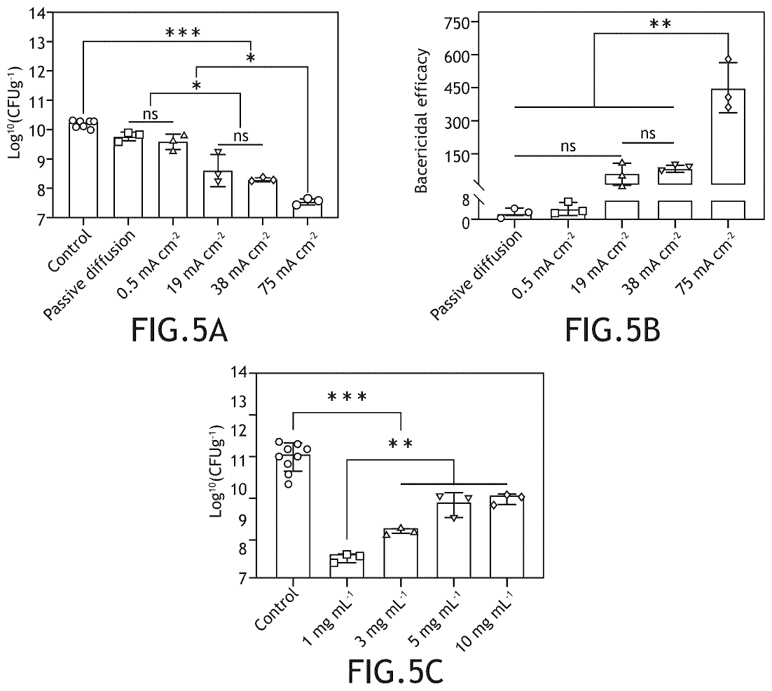

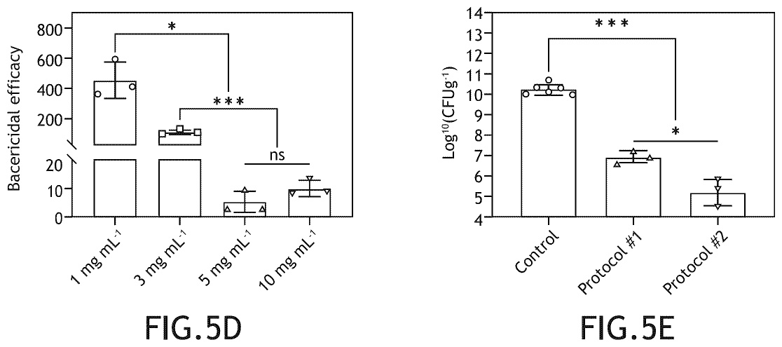

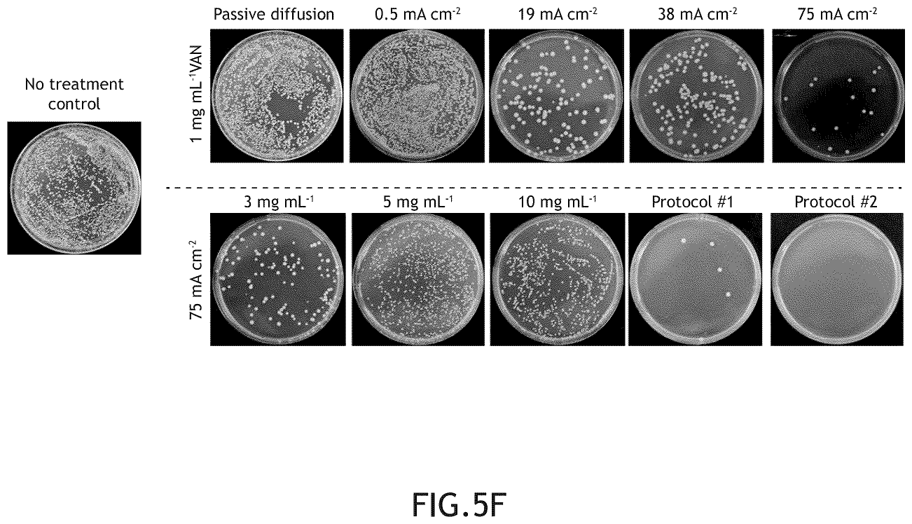

Same as previous tests, an ex vivo MRSA biofilm-infected porcine skin wound model was used here. First the biofilm treatment efficacy was tested when different current intensities were used with a fixed VAN loading concentration at 1 mg mL⁻¹. After a single treatment for 1 hour, wound tissues were incubated for 24 hours in a 37° C. incubator to allow VAN to take effect. After incubation, tissue samples were collected and evaluated for bacterial count using the standard plate counting assay for MRSA colony. As shown in FIGS. 5A and 5F, 10^{10.2} CFU g⁻¹ MRSA were measured in control wound sample receiving no treatment, which indicated the formation of a mature biofilm. Treating the biofilm with no current for 1 hour (only VAN diffusion) or a low current of 0.5 mA cm⁻² for 1 hour induced a minimal MRSA count reduction to 10^{9.8} CFU g⁻¹ and 10^{9.6} CFU g⁻¹, respectively. When 19 mA cm⁻², 38 mA cm⁻², and 75 mA cm⁻² were applied, MRSA count was further reduced to 10^{8.6} CFU g⁻¹, 10^{8.3} CFU g⁻¹ and 10^{7.6} CFU g⁻¹, respectively (see FIGS. 5A and 5F). The bactericidal efficacy of these treatments was calculated using different current intensities, which is shown in FIG. 5B. A bactericidal efficacy of 2.7±2.06 times, 4.4±2.76 times, 57.8±50.30 times, and 81.4±13.34 times was achieved for passive diffusion, 0.5 mA cm⁻², 19 mA cm⁻², and 38 mA cm⁻², respectively. 75 mA cm⁻² significantly increased the bactericidal efficacy to 452.8±115.32 times, which was equivalent to a 99.8% bactericidal rate. Because 75 mA cm⁻² had better anti-biofilm efficacy than other conditions, it was selected for the following tests.

Next, attempts were made to study the biofilm treatment efficacy of the HIC-based biofilm treatment system using 75 mA cm⁻² as the current intensity while varying the VAN loading concentration. A single treatment of 1 hour was applied. At 24 hours after the completion of the treatment, MRSA bacterial counts of wound tissues were measured. As shown in FIGS. 5C and 5F, the bacterial count for 3 mg mL⁻¹, 5 mg mL⁻¹, and 10 mg mL⁻¹ loading concentrations were 10^{8.2} CFU g⁻¹, 10^{8.8} CFU g⁻¹, and 10^{9.0} CFU g⁻¹, respectively. They were significantly higher than the bacterial count in tissue treated with 1 mg mL⁻¹ VAN loading concentration, which was 10 76 CFU g⁻¹. The bactericidal efficacy was calculated and was found to decrease from 452.8±115.32 times (1 mg mL⁻¹) to 112.6±13.58 times (3 mg mL⁻¹), 5.1±3.80 times (5 mg mL⁻¹) and 9.7±2.92 times (10 mg mL⁻¹) (see FIG. 5D). The results here showed that although a higher VAN loading concentration in the HIC-based biofilm treatment system 100 increased the concentration of VAN delivered into the wound tissue (see FIG. 4D), it did not lead to a better biofilm treatment efficacy. This phenomenon was known as a paradoxical effect, defined as a decrease in antibacterial activity of an antibiotic at higher concentrations compared to lower concentrations. It has been reported that VAN displays the paradoxical effect when treating C. difficle (a Gram-positive bacteria), where the antibacterial activity starts to decrease at 64×MIC (A. M. Jarrad, M. A. Blaskovich, A. Prasetyoputri, T. Karoli, K. A. Hansford, M. A. Cooper, Frontiers in Microbiology 2018, 9, 1420). The results in FIG. 4D show that VAN loading concentrations of 3 mg mL⁻¹, 5 mg mL⁻¹ and 10 mg mL⁻¹ all delivered more than 16 mg g⁻¹ VAN into the wound tissue when 75 mA cm⁻² was applied. It was speculated that the concentration threshold for the paradoxical effect of VAN pertaining to MRSA biofilm treatment happened somewhere between 4.5 mg g⁻¹ (VAN concentration delivered by 1 mg mL⁻¹ loading concentration) and 16.7 mg g⁻¹ (VAN concentration delivered by 3 mg mL⁻¹ loading concentration). To test this hypothesis, a study was performed to investigate the anti-biofilm efficacy of vancomycin at different concentrations from 1 mg mL⁻¹ to 20 mg mL⁻¹ after passive incubation on MRSA biofilm-infected porcine skin wound for 24 hours. Immediately after treatment, the bacterial count in the skin wound was evaluated (see FIG. 12B). The bacterial count was 10^{8.7} and 10^{8.2} CFU g⁻¹ when 1 mg mL⁻¹ and 4.5 mg mL⁻¹ vancomycin were used, respectively. This result suggested that better anti-biofilm efficacy was achieved when increasing vancomycin concentration from 1 to 4.5 mg mL⁻¹. However, further increasing vancomycin concentration to 10 mg mL⁻¹ and 20 mg mL⁻¹ led to worse anti-biofilm efficacies. The bacterial count was 10^{8.9} CFU g⁻¹ and 10^{9.2} CFU g⁻¹, for 10 mg mL⁻¹ and 20 mg mL⁻¹ vancomycin, respectively. These results provided further evidence to support to a “threshold concentration” hypothesis. Because 1 mg mL⁻¹ VAN loading concentration had better anti-biofilm efficacy than other loading concentrations, it was selected for the following tests.

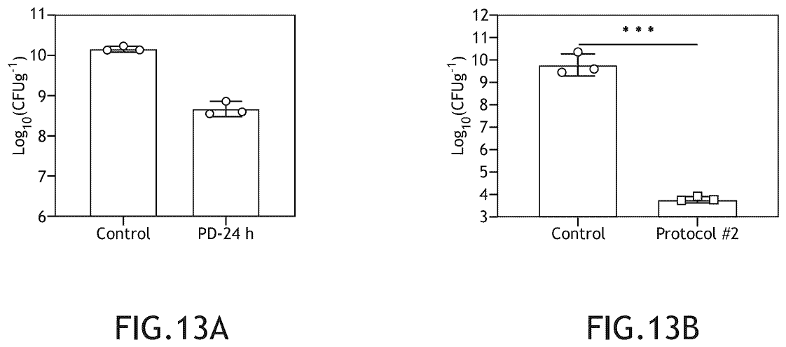

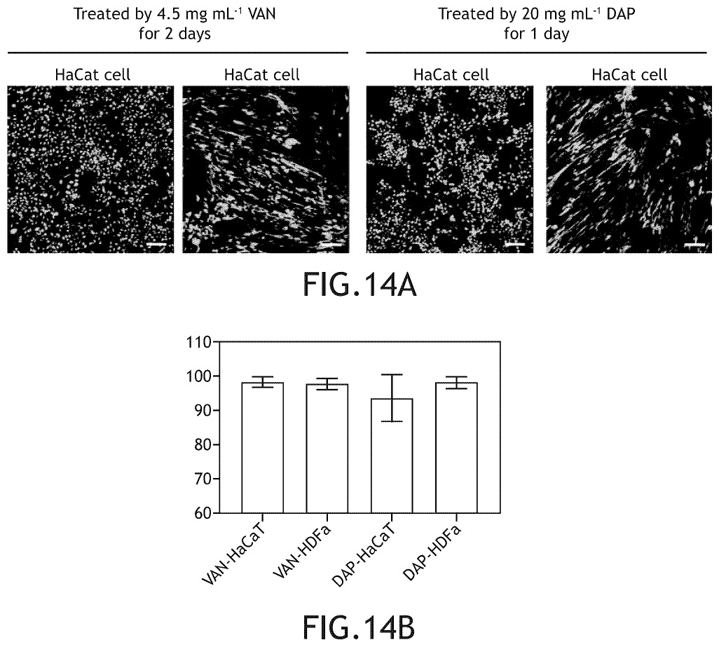

Although a single 1 hour treatment using 75 mA cm⁻² and 1 mg mL⁻¹ VAN loading concentration reduced the MRSA bacterial count in wound tissue by 2.6 login scales, the final bacterial count (10^{7.6} CFU g⁻¹) was above the clinical threshold for infection (10⁵ CFU g⁻¹). Here, it was sought to further improve the bactericidal efficacy by applying multiple treatments. Two protocols were tested. Protocol #1 applied two treatments separated by 6 hours. Protocol #2 applied two treatments separated by 24 hours. Each treatment applied 75 mA cm⁻² for 1 hour and used 1 mg mL⁻¹ VAN loading concentration. MRSA bacterial count in wound tissues was measured at 24 hours after the last treatment. As shown in FIGS. 5E and 5F, the bacterial count reduced to 10^{7.0} CFU g⁻¹ using protocol #1. When protocol #2 was used, bacterial count decreased to 10^{5.2} CFU g⁻¹. The biofilm treatment efficacy of 24 hours passive diffusion of 1 mg mL⁻¹ VAN was also tested, which mimicked the conventional long-term topical antibiotic administration used to treat chronic wound biofilm infections. 24 hours VAN diffusion only reduced bacterial count to 10^{8.7} CFU g⁻¹ measured at 24 hours after treatment (see FIG. 13A), which was inferior to both of the single and repeated treatment protocols. When protocol #2-treated wound samples were incubated for 4 days after treatment (instead of 24 hours), the number of MRSA colonies was further reduced to 10^{3.8} CFU g⁻¹ (see FIG. 13B). This was below the threshold for clinical infection and thus was considered not inhibitory to normal wound healing. It is worth noting that the viability of skin cells was not affected by 4.5 mg mL⁻¹ VAN (the VAN concentration delivered to wound tissues by the HIC-based biofilm treatment system) after 48 hour incubation (see FIG. 14A), suggesting a good cytocompatibility of the high VAN concentrations delivered by the HIC-based biofilm treatment system 100.

Findings in this section demonstrate the therapeutic efficacy of the (high-current intensity) HIC-based biofilm treatment system 100 that used a 2-hour treatment in total (protocol #2) to reduce the bacterial count in MRSA biofilm-infected porcine skin wound tissues from 10^{10.2} CFU g⁻¹ before treatment to 10^{5.2} CFU g⁻¹ at 24 hour after treatment and 10^{3.8} CFU g⁻¹ at 4-day after treatment.

Further Enhancement of Anti-Biofilm Efficacy by Combining High-Intensity Current with Daptomycin (DAP)

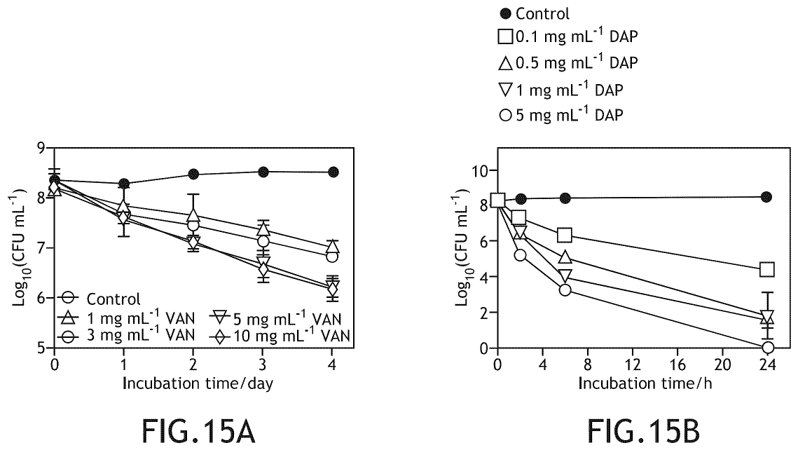

Although VAN is the standard treatment for clinical S. aureus infections, newly developed antibiotics have shown better treatment efficacy. Daptomycin (DAP), a cyclic lipopeptide antibiotic, was approved by the FDA in 2003 to treat serious infections caused by Gram-positive bacteria. Because the mode of action of DAP is less reliant on the metabolic activities of bacteria, it is considered more effective in killing metabolically inactive bacteria than conventional antibiotics, such as VAN. This is corroborated by bacteria killing studies using planktonic MRSA in stationary phase (see FIGS. 15A and 15B). The studies showed that 10 mg mL⁻¹ VAN reduced bacterial count from 10⁸ CFU g⁻¹ to 10⁶ CFU g⁻¹ after a 4-day incubation (see FIG. 15A), while 5 mg mL⁻¹ DAP reduced bacterial count from 10⁸ CFU g⁻¹ to 0 CFU g⁻¹ within 24 hours (see FIG. 15B).

In light of the superb MRSA killing efficacy of DAP, this section explores the use of DAP to further enhance the anti-biofilm efficacy of the HIC-based biofilm treatment system 100. Since DAP is negatively charged at physiological pH, it was loaded in the drug/buffer chamber of the cathode device, which was used as the working device in all the tests discussed in this section. An ex vivo MRSA biofilm-infected porcine skin wound model was used in all tests in this section.

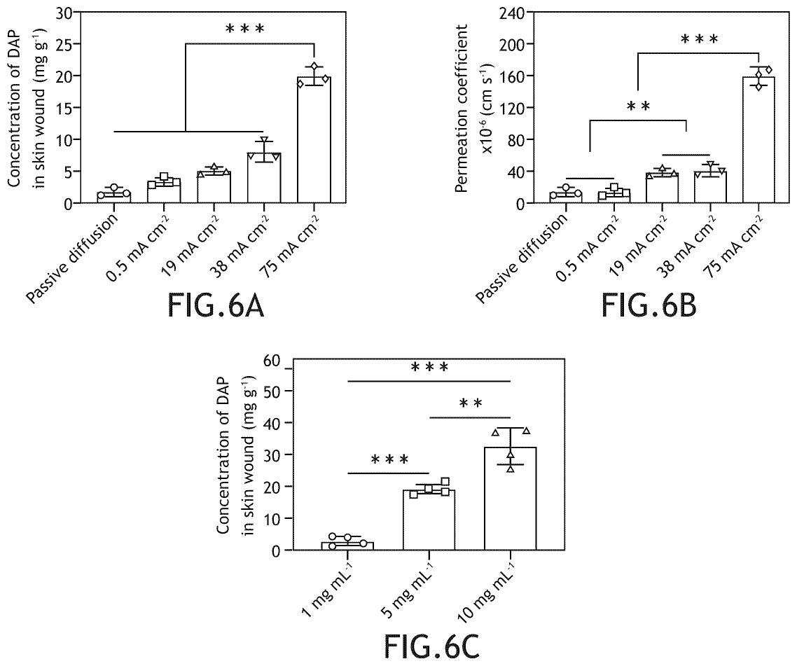

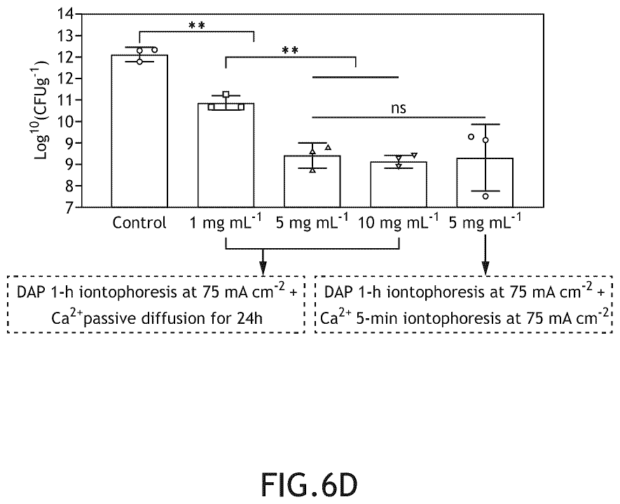

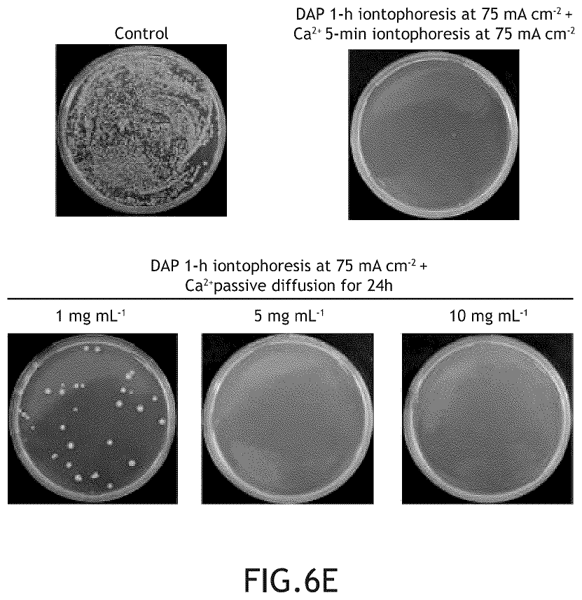

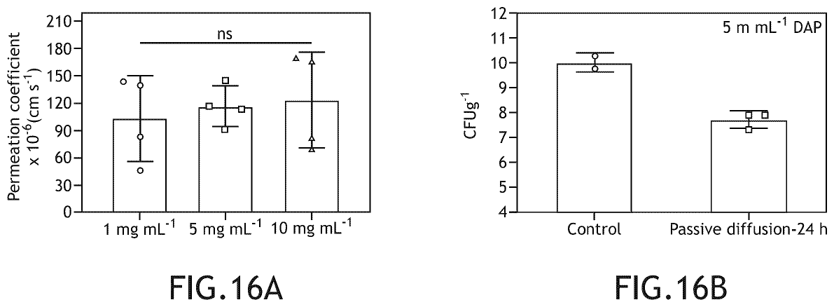

First the iontophoretic delivery efficiency of DAP was evaluated at different current intensities and DAP loading concentrations. As shown in FIG. 6A, the concentration of DAP delivered to wound tissues after 1 hour iontophoresis was significantly increased with increasing current intensity, from 1.7±0.59 mg g⁻¹ by passive diffusion to 19.9±1.45 mg g⁻¹ by 75 mA cm⁻². The Pc of DAP for 75 mA cm⁻² iontophoresis reached 158.3±11.18×10⁻⁶ cm s⁻¹, which was 12.0 times higher than passive diffusion (Pc=13.2±3.15×10⁻⁶ cm s⁻¹) and 10.1 times higher than 0.5 mA cm⁻² iontophoresis (Pc=15.7±3.37×10⁻⁶ cm s⁻¹) (see FIG. 6B). The concentration of DAP delivered to wound samples after 1 hour iontophoresis also had a linear relationship with the DAP loading concentration. As shown in FIG. 6C, when 1 mg mL⁻¹ DAP was loaded in the working device, 75 mA cm⁻² iontophoresis delivered 3.3±1.49 mg g⁻¹ DAP into the wound tissue. When 5 mg mL⁻¹ and 10 mg mL⁻¹ DAP was loaded, the DAP concentration delivered into wound tissue increased to 19.9±1.45 mg g⁻¹ and 32.8±5.68 mg g⁻¹, respectively. The Pc of DAP at different loading concentrations was calculated and was found to be independent of loading concentration (see FIG. 16A). This result suggested that unlike VAN, DAP did not exhibit concentration saturation effect.

Since 75 mA cm⁻² produced the highest iontophoretic delivery efficiency for DAP, it was used as the current intensity in the following anti-biofilm efficacy study. Three DAP loading concentrations (1, 5 and 10 mg mL⁻¹) were tested in the anti-biofilm efficacy study. After a single 1 hour treatment, wound tissues were incubated in a 37° C. incubator for 24 hours. During incubation, calcium chloride solution was topically applied to the wound, because DAP required calcium ions to function. After incubation, bacterial count in wound tissues was measured. As shown in FIG. 6D and FIG. 6E, MRSA bacterial count was reduced from 10^{10.1} CFU g⁻¹ (untreated control) to 10 79 CFU g⁻¹, 10^{5.4} CFU g⁻¹ and 10^{5.2} CFU g⁻¹ when 1 mg mL⁻¹, 5 mg mL⁻¹ and 10 mg mL⁻¹ DAP was loaded in the drug/buffer chamber, respectively. The final bacterial count achieved by 5 mg mL⁻¹ DAP loading concentration reached the clinical threshold for infection and was same as the bacterial count achieved by VAN treatment protocol #2 (measured at 24 hours post treatment; see FIG. 5E). In comparison, when the same biofilm-infected wound tissue was treated with topically applied 5 mg mL⁻¹ DAP mixed with calcium chloride solution (passive diffusion) for 24 hours, the bacterial count was only reduced to 10^{7.7} CFU g⁻¹ (see FIG. 16B). Similar to VAN, 20 mg mL⁻¹ DAP (the DAP concentration delivered to wound tissues by 5 mg mL⁻¹ loading concentration) did not have significant impact on the viability of skin cells after a 24 hour incubation (see FIGS. 14A and 14B), suggesting a good cytocompatibility of the high DAP concentrations delivered by the HIC-based biofilm treatment system 100.

Although efficacious, the foregoing treatment protocol requires a long total treatment duration including a 1 hour electrical treatment by the HIC-based biofilm treatment system followed by a 24 hour topical application of calcium chloride solution.

To further reduce the total treatment duration, a 5 minute anodal iontophoresis at 75 mA cm⁻² was used to introduce calcium ions into the wound tissue immediately after the 1 hour treatment with 5 mg mL⁻¹ DAP loading concentration. The anodal iontophoresis of calcium ions was applied by the HIC-based biofilm treatment system 100. Wound samples were then incubated for 24 hours without topical calcium chloride solution before bacterial counting. As shown in FIGS. 6D and 6E, this new protocol achieved a similar bacterial count reduction to 10^{5.3} CFU g⁻¹ as the previous protocol, while only requiring a 65 minute total treatment duration.

In summary, the results in this section show that by using DAP, a 65 minute high-intensity electrical treatment can achieve the same anti-biofilm efficacy as a 2 hour treatment using VAN.

In vivo Safety of the HIC-Based Biofilm Treatment System

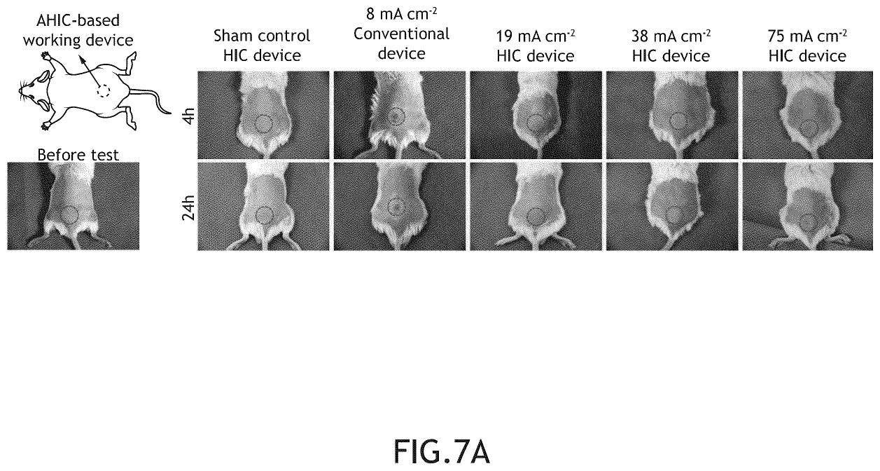

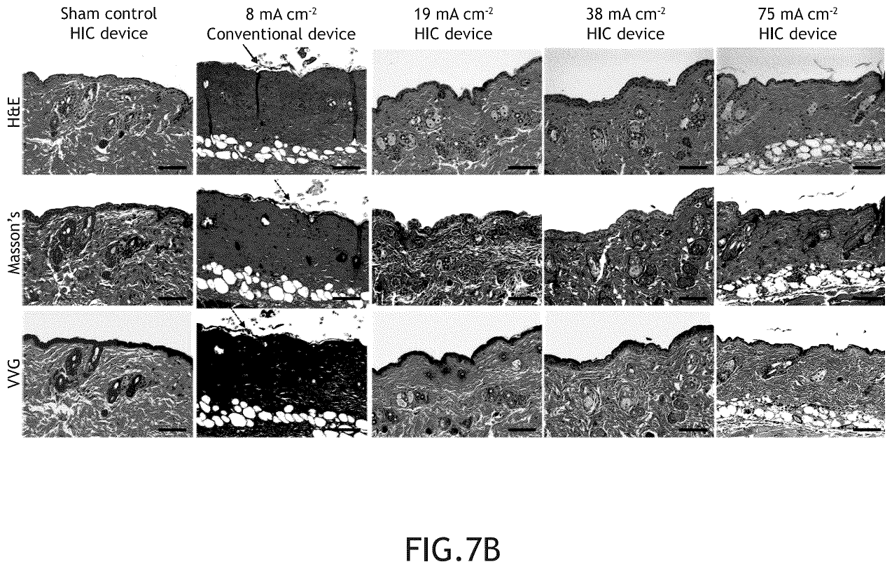

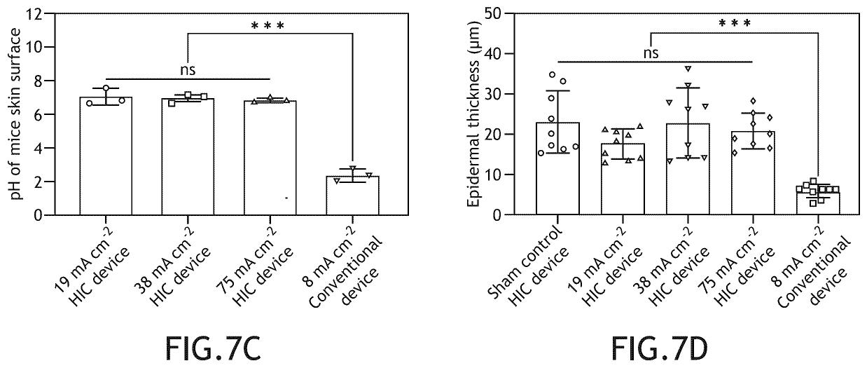

In previous sections, the in vitro and ex vivo safety of the HIC-based biofilm treatment system 100 was demonstrated by focusing on pH and temperature stability and cell viability. This section goes a step further to evaluate the in vivo safety of the HIC-based biofilm treatment system 100, which is a critical prerequisite for therapeutic efficacy evaluation of the HIC-based biofilm treatment system 100 using animal models and eventually in human patients. Healthy mice were used in this test. The working device 104 (anode) was attached to the lower back of the mouse along the midline (see FIG. 7A). The counter device 106 was also attached to the back of the mouse, located 1 cm away from the working device 104. The effective treating area with skin was 0.125 cm². The drug/buffer chambers 114 and 122 of both working and counter devices 104 and 106, respectively, were filled with 1x PBS. Since anode device always represents a worst-case scenario in terms of tissue damage, only skin tissue samples that were in contact with the anode device were evaluated.

Different current intensities, including 0 mA cm⁻² (sham control), 19 mA cm⁻², 38 mA cm⁻², and 75 mA cm⁻², were applied by the HIC-based biofilm treatment system 100 for 1 hour. A conventional electrical device was constructed by inserting a carbon electrode into a PBS-filled drug/buffer chamber with the same volume as the HIC-based biofilm treatment system (see FIG. 2B). The current intensity was gradually increased from 0.5 mA cm⁻² to identify the minimum current that induced significant skin tissue damage after 1 hour application when the conventional device was used to apply the current. Human clinical trials showed that conventional low-intensity iontophoresis typically induced mild skin irritation and erythema that rarely lasted more than 3 hours after the removal of iontophoresis. Therefore, it was chosen to follow up at 4 hours and 24 hours after treatment for skin damage evaluation.

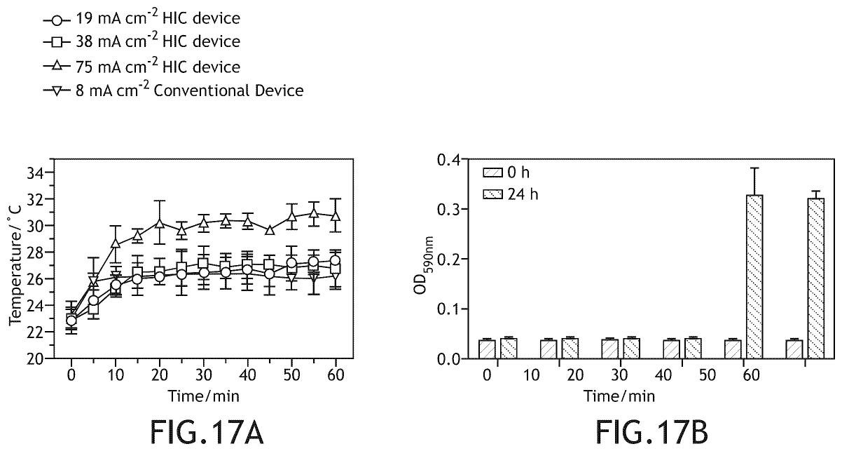

As shown in FIG. 7A, no signs of skin irritation, redness or blisters were observed in mice treated with the HIC-based biofilm treatment system 100 for all current intensities tested at both 4 hour and 24 hour checkpoints. However, the skin tissue treated with the conventional device started to show obvious redness and irritation in device contact area at 8 mA cm⁻², which did not recover by 24 hours. The skin surface pH in the current treating area was also measured immediately after different treatments. FIG. 7C shows that the pH of the skin treated by the HIC-based biofilm treatment system remained in the safe range for all current intensities tested (pH=7.1±0.52, 7.0±0.20, and 6.9±0.11 for 19 mA cm⁻², 38 mA cm⁻², and 75 mA cm⁻² treatments, respectively). However, the pH of the skin treated by conventional device at 8 mA cm⁻² decreased to 2.38±0.38. This was mainly due to the accumulation of hydrogen ions generated by EC reactions, which was not sufficiently neutralized by the pH buffering system used in the conventional device (i.e., PBS). This acidic environment caused the skin irritation that can be seen in FIG. 7A. Similar skin tissue damage caused by pH-induced chemical burn was also reported by other studies (M. Kim, H. Kim, H. W. Kang, Lasers in surgery and medicine 2018, 50, 661; J. Macêdo, J. A. Ferreira Júnior, K. A. Nascimento, M. S. Lacerda, N. E. Pereira, P. M. Pedroso, Pesquisa Veterinária Brasileira 2018, 38, 2088). The skin surface temperature was also measured immediately after treatment, which remained below 43° C. in all groups (see FIG. 17A). This result suggested that pH change was the major cause for skin damage in conventional device-treated animals. Histological sections of the skin tissues in direct contact with the HIC-based biofilm treatment system or the conventional device were collected at 24 hours after treatment (see FIG. 7B). In tissues treated by conventional device at 8 mA cm⁻², fluid accumulation and swelling of collagen bundles in the dermis layer were observed. These resulted in missing cleft spaces between collagen bundles, homogenization and full-thickness hyalinized necrosis of the dermal collagens. The epidermis in conventional device-treated samples demonstrated significant thinning with keratinocytes necrosis and focal detachment as compared to the sham control. In contrast, although mild neutrophil infiltration was observed, there was no significant epidermal or dermal damage in skin tissues treated by the HIC-based biofilm treatment system 100. The epidermal thickness was measured for all samples, and it was found that treatment with the HIC-based biofilm treatment system 100 did not significantly alter the epidermal thickness in all current intensity treatment groups (see FIG. 7D). However, conventional device treatment at 8 mA cm⁻² significantly reduced the average epidermal thickness by more than 50% compared to sham control. This result was consistent with histological observations. The outcome of this study demonstrated that the HIC-based biofilm treatment system 100 significantly enhanced the in vivo safety of high-intensity current application to mouse skin tissues compared to the conventional electrical device.

In vivo Anti-Biofilm Efficacy of the HIC-Based Biofilm Treatment System

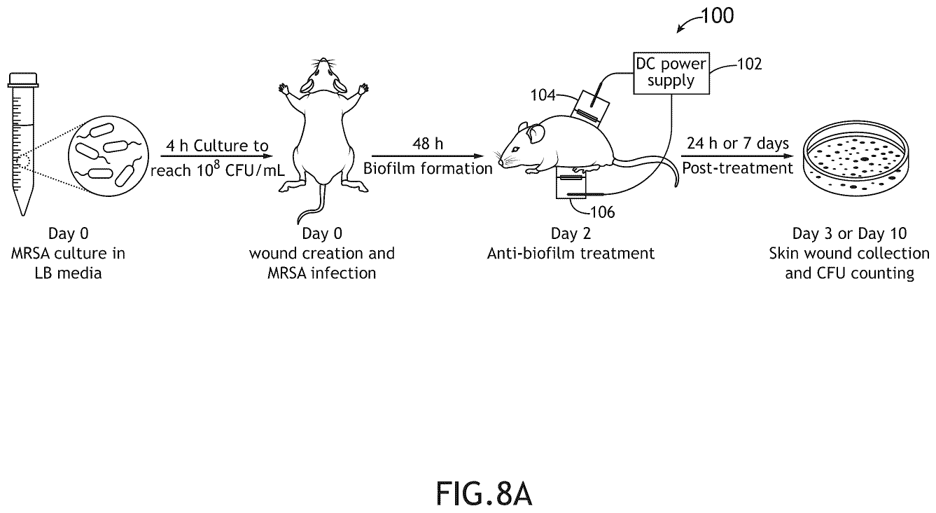

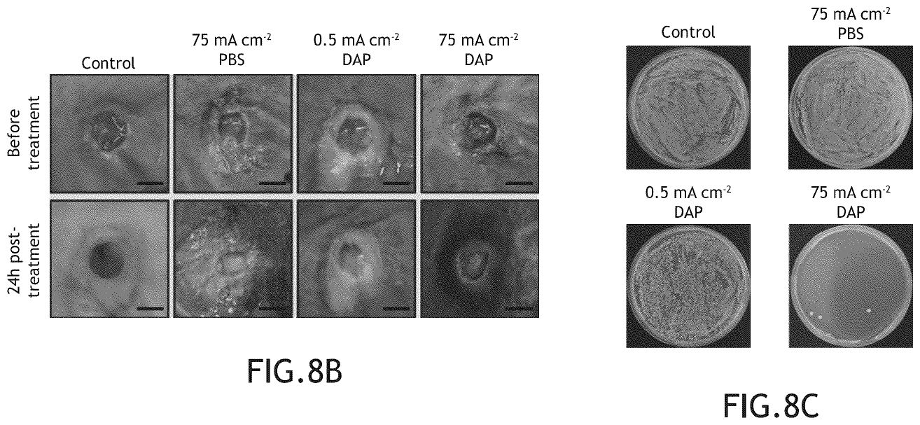

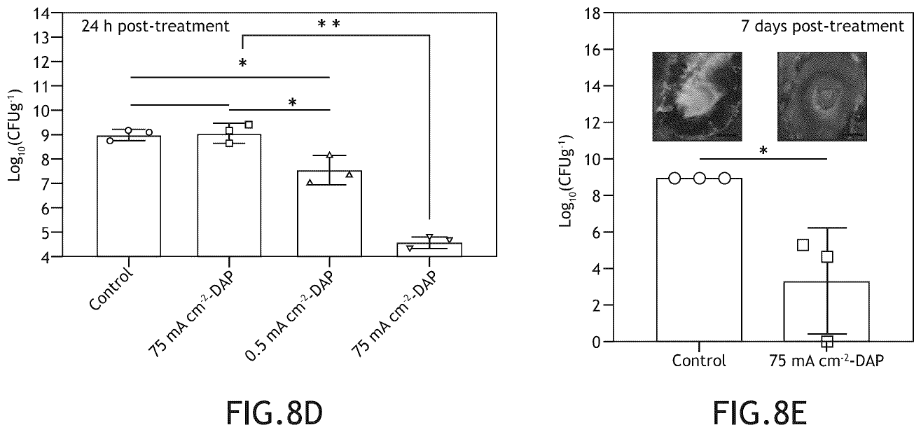

The in vivo anti-biofilm efficacy of the HIC-based biofilm treatment system 100 has also been investigated using a type II diabetic mouse-based skin wound model infected with the MRSA biofilm. The wound infection model was established following previously published protocols with modifications. In brief, full-thickness wounds were created with a 4 mm biopsy punch on the back of the diabetic mouse. MRSA was cultured for 4 hours to reach 1×10⁸ CFU mL⁻¹ in vitro, and then inoculated into the wound site. Two days after wound inoculation, mature biofilms were formed on the wound. Daptomycin was used in this in vivo anti-biofilm efficacy study because it required a shorter treatment duration to achieve the same efficacy as vancomycin in the ex vivo studies. As shown in FIG. 8A, which illustrates the experimental procedure, the working device 104 was attached to the wound site on the back of the mouse. The counter device 106 was attached to the belly directly below the working device. The effective treating area with skin wound was 0.125 cm², which can entirely cover the wound site. Four different treatments were tested, including untreated negative control, high-intensity current alone (75 mA cm⁻² was applied for 60 minutes, drug/buffer chamber was loaded with PBS), low-intensity current (0.5 mA cm⁻² was applied for 60 minutes) with DAP (5 mg mL⁻¹ was loaded in the drug/buffer chamber), and high-intensity current (75 mA cm⁻² was applied for 60 minutes) with DAP (5 mg mL⁻¹ was loaded in the drug/buffer chamber). For all treated groups, iontophoresis of calcium ions was applied for 5 minutes at 75 mA cm⁻² after the 60 minute treatment. The biofilm bacterial count was measured at 24 hours or 7 days after the treatment by using standard plate counting assay. FIG. 8B shows representative photographs of the biofilm infected diabetic wound immediately before and at 24 hours after the treatment. FIG. 8C shows the bacterial colony cultured from wound samples collected at 24 hours after treatment. The quantitative measurement of bacterial count in wound samples collected at 24 hours after treatment is graphically presented in FIG. 8D. The bacterial count in no-treatment control group reached 10^{9.0} CFU g⁻¹ at Day 3. The high-intensity electrical treatment alone did not reduce the bacteria density, which remained at 10^{9.1} CFU g⁻¹. This result was consistent with the ex vivo electrical debridement study, which showed that biofilm recovery happened within 24 hours after the treatment. Low current intensity combined with DAP only reduced the bacterial density from 10^{9.0} CFU g⁻¹ to 10^{7.6} CFU g⁻¹, which was above the clinical threshold for wound infection. In contrast, when high-intensity current combined with DAP was applied, the bacterial count in the wound at 24 hours after treatment reduced to 10^{4.6} CFU g⁻¹, which was below the clinical threshold for wound infection. The bacterial count at 7-day after the treatment of high-intensity current with DAP further decreased to 10^{3.3} CFU g⁻¹, which suggested that the high DAP concentration delivered by the HIC-based biofilm treatment system 100 exhibited a sustained anti-biofilm effect. The outcome of this study was significant because it showed that in vivo wound biofilm infections can be effectively reduced by a one-time, short-duration electrical treatment using the HIC-based biofilm treatment system 100. This treatment efficacy achieved by the HIC-based biofilm treatment system 100 was better or same as the efficacy achieved by prolonged, continuous application of antibiotics on biofilm for 24 hours or longer using wound dressings or microneedle devices. A much shorter treatment duration would be particularly advantageous for management of chronic wound infections as it would reduce patient discomfort and enhance patient compliance.

### Potential Improvements

In studies, the HIC-based biofilm treatment system 100 did not completely eradicate the biofilm (i.e., the biofilm bacterial count was not reduced to 0 CFU g⁻¹). However, the biofilm treatment efficacy of this technology is likely limited by the antibiotics used in the HIC-based biofilm treatment system 100. It has been reported that VAN and DAP are not capable of killing S. aureus persister cells even at 100×MIC (W. Kim, G. Zou, T. P. Hari, I. K. Wilt, W. Zhu, N. Galle, H. A. Faizi, G. L. Hendricks, K. Tori, W. Pan, Proceedings of the National Academy of Sciences 2019, 116, 16529). The development of new anti-biofilm agents that are more effective against persister cells is an active research area. More efficacious anti-biofilm drugs will undoubtedly enhance the biofilm treatment efficacy of this technology. Delivering a combination of anti-biofilm agents instead of a single one may further enhance the efficacy of this technology. EPS degrading agents and metabolic adjuvants have been shown to enhance biofilm bacterial killing of antibiotics. Anti-inflammatory agents can reduce tissue damage induced by biofilm infections. By applying high-intensity current and combining with multi-drugs using the HIC-based biofilm treatment system 100, it is contemplated that a greater anti-biofilm treatment efficacy can be achieved. Although it was demonstrated that the HIC-based biofilm treatment system 100 had enhanced safety compared to the conventional electrical device, a more comprehensive study will need to be performed in the future to further and fully characterize the safety of the high-intensity current applied by the HIC-based biofilm treatment system 100, including the evaluation of integrity and function of skin cells and tissues, pain sensation, and neuromuscular functions.

### Conclusion

The HIC-based biofilm treatment system 100 is a novel electrical current-based biofilm treatment system for combating chronic wound biofilm infections. This disclosure demonstrates the safety, the electrical biofilm debridement efficacy, and the iontophoretic antibiotic delivery efficacy of the HIC-based biofilm treatment system 100 using high current intensities of up to 75 mA cm⁻². By combining high-intensity electrical debridement and high-efficacy iontophoretic antibiotic delivery, the HIC-based biofilm treatment system 100 used a short treatment (e.g., less than 2 hours) to successfully reduce the bacterial count of mature biofilm-infected skin wounds ex vivo and in vivo to below the clinical threshold for wound infection. The innovative technology provides a simple, quick, safe, yet highly efficacious means to manage biofilm infections in chronic wounds. The reduction of bacterial bioburden will help resume the normal healing process in chronic wounds. Ultimately, this will reduce the amputation rate related to chronic wounds, enhance patients' quality of life, and reduce the overall healthcare cost.

### Materials and Methods

The sections below provide descriptions and examples of materials and methods employed to conduct the (previously discussed) studies and testing that were used to validate the safety and efficacy of the HIC-based biofilm treatment system 100.

### Materials

Polyethylene glycol dimethacrylate (PEGMDA, molecular weight=8000) was purchased from Polysciences (Warrington, Pa., USA). Poly(ethylene glycol) diacrylate (PEGDA, molecular weight=700), IRGACURE 2959, Benzophenone, Fluorescein isothiocyanate (FITC) labeled dextran-4 kDa (FD-4), water with 0.1% (v:v) trifluoroacetic acid (TFA), acetonitrile, acetonitrile with 0.1% (v:v) TFA, Agar, sodium phosphate monobasic (NaH₂PO₄), and sodium phosphate dibasic (Na₂HPO₄) were purchased from Sigma-Aldrich (St. Louis, Mo., USA). LB Broth (Miller) was purchased from Fisher Bioreagents (Fair Lawn, N.J., USA). Acrylic sheets and very-high-bond (VHB) foam tape were purchased from Mcmaster-Carr (Robbinsville, N.J., USA). Fetus porcine skin was purchased from Nebraska Scientific (Omaha, Nebr., USA). Human keratinocyte cells (HaCaT), human primary dermal fibroblast cells (HDFa), and human monocytic cells (U937) were kind gifts from Dr. Jingwei Xie at the University of Nebraska Medical Center (Omaha, Nebr., USA).

### Bacterial Strain, and Antibiotics

Methicillin-resistant S. aureus (MRSA) USA300 strain was used in this study. The planktonic bacteria were cultured in LB medium. Vancomycin hydrochloride (Apexbio Technology LLC, Houston, Tex., USA) and daptomycin (Combi-Blocks, San Diago Calif., USA) were used to treat MRSA biofilm. The minimum inhibitory concentration (MIC) of vancomycin hydrochloride to planktonic MRSA cells was determined as 1.2 μg mL⁻¹ (see FIG. 17B), which was in consistent with the results reported previously.

### Fabrication of HIC-Based Electrical Biofilm Treatment System

The HIC-based biofilm treatment system 100, including working device 104 and counter device 106, was fabricated by laser micromachining (Trotec Speedy 300, Trotec., Mich., USA). The drug/buffer chambers 114 and 122 and the phosphate salt solution chambers 110 and 118 were fabricated using acrylic plastic. To assemble different components of device, a double-adhesive VHB tape was used. PEG hydrogels 112 and 120 were composed of 10% PEGDMA, 5% PEGDA and 1% Irgacure 2959 and 84% deionized water. The hydrogels 112 and 120 were bonded to the phosphate salt solution chambers 110 and 118, respectively, to form a two-phase separation system (ATPS) by using UV lamp. Benzophenone (10% w/v) was used to allow the binding. To obtain phosphate salt solutions with high conductivities, saturated Na₂HPO₄ solution (0.6 mol L⁻¹, pH=9.0, 46.1±2.40 ms cm⁻¹) was used in anode HIC-based device. And a mixture solution containing NaH₂PO₄ (0.6 mol L⁻¹) and Na₂HPO₄ (0.48 mol L⁻¹) (pH=6.4, 51.0±0.51 ms cm⁻¹) was used in cathode HIC-based device. The working device 104 and counter device 106 of the HIC-based biofilm treatment system 100 were connected with the DC power supply 102 using carbon electrodes 108 and 116, respectively.

In vitro Safety Test of High-Intensity Current Application and High Concentration of Antibiotics

The in vitro cell viability test setup of high-intensity ion current has been described in previous work. Briefly, two adhering cells, HaCaT cells (human keratinocyte cell line) and HDFa cells (human primary dermal fibroblast cells), were seeded in 8 mm×8 mm areas in cell culture dishes defined by a PDMS stencil, and cultured overnight. After removing the PDMS stencils the next day, the test device with a rectangular fluidic chamber was amounted on the cell culture dish, so the current density adhering cells experienced can be precisely defined. After adding complete growth media, 75 mA cm⁻² DC current intensity was conducted for 1 hour. After test, cell counting assay was used to count the live cells. Human monocytic cells (U937) were also used to evaluate the cell viability after high current intensity treatment. They were cultured overnight in complete growth media and transferred to the test device chamber the next day. Cells with no electrical current treatment were used as control group for cell viability comparison.

To evaluate the cell toxicity of the high concentration of antibiotics, HaCaT cells and HDFa cells were used. 2×10⁴ HaCat cells and 2×10³ HDFa cells were seeded in each well of a 96-well plate. After being cultured in an incubator under 37° C. and 5% CO₂ for 24 hours, 4.5 mg mL⁻¹ VAN and 20 mg mL⁻¹ DAP were added to the culture media, respectively. Both HaCat cells and HDFa cells were treated by VAN for two days, and exposed to DAP for one day, respectively. The cell toxicity of the two cells at the end time points were evaluated by LIVE/DEAD stain and visualized by a confocal laser scanning microscope (CLSM, LSM 710, Zeiss, Germany).

In vitro Bactericidal Effect of MRSA Planktonic Bacteria by High-Intensity Current

MRSA bacteria was cultured with LB media in a 37° C. water shaking bath for 4 hours to reach the density of 10⁸ cells mL⁻¹. Then MRSA solution was added to a rectangular fluidic chamber. After immersing the HIC-based anode device and cathode device in the bacteria solution located at two sides of the chamber, 75 mA cm⁻² DC current intensity was conducted for 1 hour. The viable bacteria densities before, immediately after treatment, and 24 hours after treatment were measured by standard plating counting assay.

Ex vivo Safety Test of High-Intensity Current Application

Freshly preserved fetal pig skin samples purchased from Nebraska Scientific (Omaha, Nebr., USA) were used for the ex vivo safety test of high current intensity applied by the HIC-based biofilm treatment system 100. Skin samples were stored at −20° C. upon arrival and used within 1 month. Skin samples were sandwiched between drug/buffer chamber 114 of the working device 104 and the PBS chamber 122 of the counter device 106 which was attached to the back of the skin sample. Before testing, the drug/buffer chamber 114 was filled with PBS. The high concentration phosphate salt solutions were added into phosphate salt solution chambers 110 and 118 of the working and counter devices 104 and 106, respectively. 75 mA cm⁻² current intensity was conducted from the DC power supply 102 and applied on the skin sample for 1 hour continuously. By using a K-type thermocouple (Digi-Sense™ Standard Precalibrated Thermocouple, Vernon Hills, Ill., USA), the peak temperature of device/tissue interface during current application was monitored. After the test, the pH of drug solution and drug/buffer chamber contacting-skin area were measured using a flat pH probe (Sensorex Corporation, Stanton, Calif.). For comparison, carbon electrode directly inserted into the drug/buffer chamber and applied same current conditions was used as the conventional control (i.e., the conventional device in FIG. 2B).

Establishment of ex vivo MRSA Biofilm Infected Skin Wound

The fetal pig skin samples (approximately 30×30 mm) were immersed in 10% (v/v) bleach for 5 minutes after hair removal. Then, skin samples were sterilized in 70% ethanol for 30 minutes. The surface of skin sample was further cleaned by 10% povidone-iodine and 70% isopropanol to prevent contamination. A 6-mm disposable biopsy punch was used to create an excisional wound. Samples were put into sterile 10-cm petri-dishes filled with sterile PBS (6 mL). The wound was then inoculated with 1×10⁸ CFU mL⁻¹ MRSA bacteria solution (20 μL). The bacteria were allowed to grow in a 37° C. Heratherm™ compact microbiological incubator (ThermoFischer Scientific™, Waltham, Mass., USA) for 48 hours to form mature biofilms.

Electrical Debridement Treatment of High-Intensity Current on ex vivo MRSA Biofilm Infected Skin Wounds

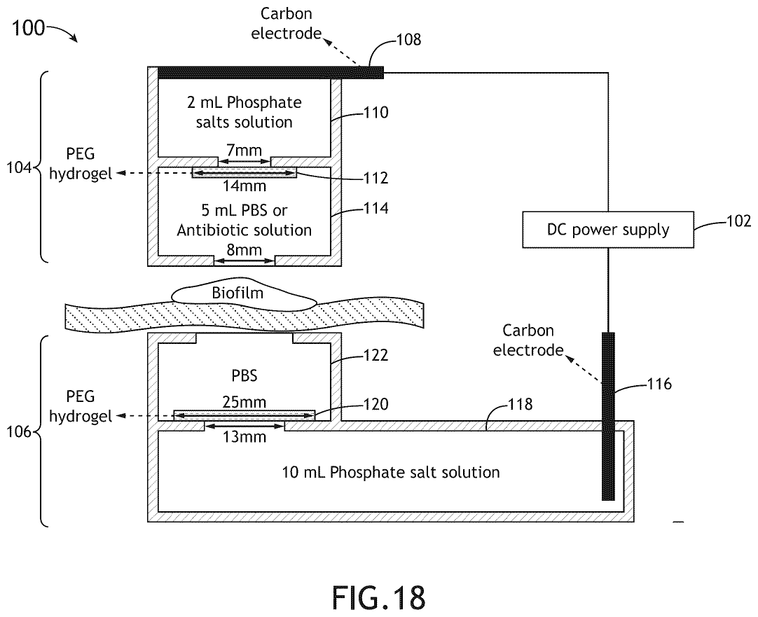

The HIC-based biofilm treatment system 100 embodiment shown in FIG. 18 was used to test electrical debridement treatment of high-intensity current on ex vivo MRSA biofilm infected skin wounds. The MRSA biofilm infected skin wound was sandwiched between anode and cathode HIC-based devices (i.e., the working device 104 and the counter device 106). To evaluate the anti-biofilm effect of high current intensity only, PBS (5 mL) was filled in both drug/buffer chamber 114 and PBS chamber 122 and then 0.5 mA cm⁻², 19 mA cm⁻², 38 mA cm⁻², and 75 mA cm⁻² was applied for 1 hour, respectively. The device/skin wound contact area was 0.502 cm². Skin samples were collected by 8-mm biopsy punch immediately after test or at 24 hours after testing. To evaluate the biofilm thickness before and after different treatments, samples were embedded in tissue freezing medium (OCT) and sectioned at 8 μm. After being imaged in microscope, the thickness of biofilms after different treatments were measured using ImageJ software. To evaluate the colony-forming unit (CFU) counts per gram of MRSA from infected skin wounds, samples were homogenized in PBS (10 mL) and diluted with PBS, and then transferred to standard plate counting assay. The bactericidal efficacy was calculated as follows:

$$Bactericidal efficacy = \frac{CFU counts per gram in initial mature biofilm}{CFU counts per gram residual in treated infected wounds} \qquad ( 1 )$$

Iontophoresis of Antibiotics Induced by the HIC-based Biofilm Treatment System to ex vivo MRSA Biofilm Infected Skin Wound

To evaluate the antibiotic delivery efficacy of VAN by high-intensity current, biofilm infected fetal pig skin wound was used. The test setup was the same as the HIC-based biofilm treatment system 100 embodiment shown in FIG. 18. The DAP test setup was same as that of VAN, except no PBS chamber 122 was used. Instead, the inner surface of the skin wound contacted directly to the hydrogel 120 of the counter device 106. To evaluate the effect of electrical current intensity on the drug delivery efficacy, different current intensities from 0 to 75 mA cm⁻² were applied. To evaluate the effect of loading concentration of antibiotics, different concentrations of VAN and DAP from 1 mg mL⁻¹ to 10 mg mL⁻¹ were used. Since VAN is neutral and DAP is negatively charged under physiological environment, anode HIC-based device was placed facing biofilm for VAN iontophoresis, and cathode HIC-based device was placed facing biofilm for DAP iontophoresis. Samples were collected immediately after different treatments. The concentrations of antibiotics delivered into skin wound samples were measured by high-performance liquid chromatography (HPLC). The permeation coefficient (Pc, cm s⁻¹) was then calculated (Pc=J/C, where J is antibiotic delivery flux in tissue samples (μg (cm²s)⁻¹, C is the loading concentration of antibiotic solution (μg m L⁻¹)).

### Quantitative Measurement of Antibiotic Concentrations in Infected Skin Wounds Using High-Performance Liquid Chromatography (HPLC)

Immediately after iontophoresis, the infected skin wounds were collected by an 8-mm disposable biopsy punch and washed in PBS three times to remove surface antibiotics, and then weighted before use. Samples were homogenized in 4-10 mL PBS and then centrifuged (4000 rpm, 20 minutes). The supernatant (200 μL) was collected and mixed with methanol or acetonitrile to remove potential tissue proteins. After solvent entirely evaporated by a hot plate (85° C., 4 hours), samples were reconstituted in optimized solution (200 μL) to restore original volume. Centrifuge (10,000 rpm) was performed for 5 minutes, and the supernatant was injected for analysis. For VAN, the optimized solution to re-dissolve samples was the mixture of water with 0.1% trifluoroacetic acid (TFA) and acetonitrile with 0.1% TFA (50:50). For DAP, samples were re-dissolved in the mixture of buffered solution (Monosodium phosphate: 188.8 mmol L⁻¹, Disodium phosphate: 11.16 mmol L⁻¹, pH=5.5) and acetonitrile (50:50).

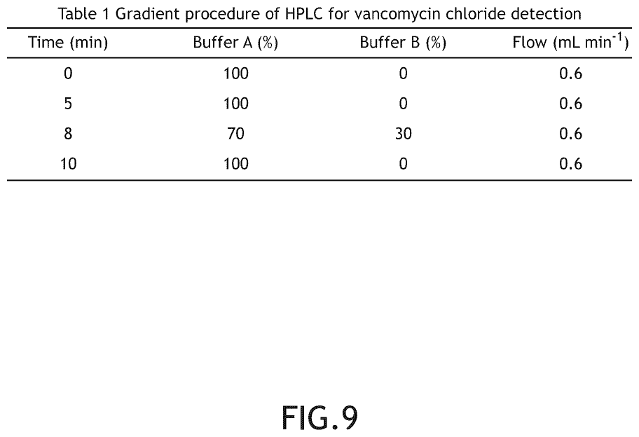

The concentrations of antibiotics accumulated in infected skin wounds were detected by HPLC on an Agilent 1260 Infinity system (Agilent, Santa Clara, Calif., USA) using an EC-C18 column (150 mm×4 mm, particle size 2.7 μm). The injection volume was 20 μL with a flow rate of 0.6 mL min⁻¹. For VAN concentration detection, a gradient procedure was used for precisely detection. The mobile phase was water with 0.1% TFA (Buffer A) and acetonitrile with 0.1% TFA (Buffer B). The gradient procedure was developed in accordance with Table 1 of FIG. 9.

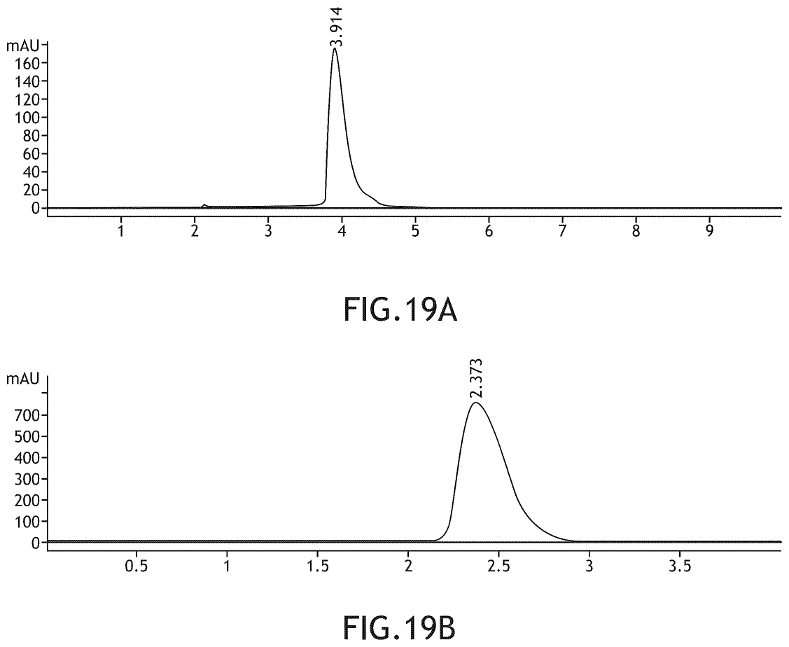

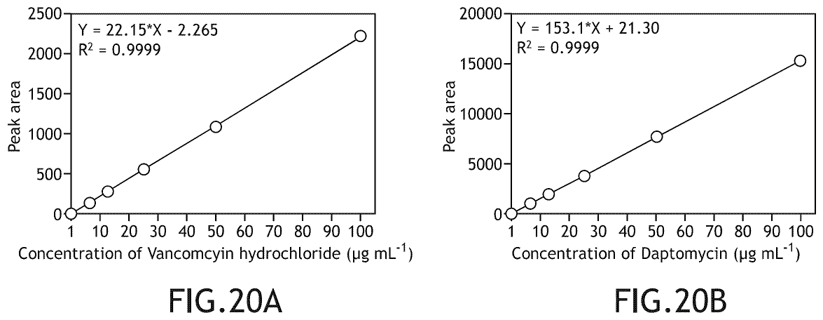

The detection was performed at 284 nm. Retention time of VAN was around 4 minutes (see FIG. 19A). For DAP concentration detection, the mobile phase was buffered solution (Monosodium phosphate: 188.8 mmol L⁻¹, Disodium phosphate: 11.16 mmol L⁻¹, pH=5.5) and acetonitrile with ratio of 50:50 (v/v). The detection was performed at 223 nm. The retention time of DAP was around 2.3 minutes (see FIG. 19B). Accumulated concentrations of VAN and DAP were calculated via a standard calibration curve of VAN and DAP ranging from 25 μg mL⁻¹ to 100 μg mL⁻¹, respectively (see FIGS. 20A and 20B).

### Cryo-Section and Fluorescent Microscopy

FD-4 penetration and distribution in the MRSA biofilm infected skin wound was evaluated by cryo-section and fluorescent microscopy. Briefly, samples were collected by 8-mm disposable biopsy punch and dehydrated in acetone for 10 to 30 minutes. After being air dried for another 10 to 30 minutes, the samples were embedded in tissue freezing medium (OCT) and sectioned to obtain 10 μm section under −20 ° C. Section samples were then observed under a fluorescent microscope (DMI 6000 B, Leica, Bannockburn, Ill., USA).

### Minimum Inhibitory Concentration (MIC) and Time-Killing Tests of Antibiotics to Planktonic MRSA

MRSA bacteria was cultured in LB media in a 37° C. water shaking bath overnight to reach the concentration of 1×10⁸ CFU mL⁻¹. Then, they were diluted to 10⁵ CFU mL⁻¹ in 96-well plates by adding different concentrations of VAN solutions. After incubation for 24 hours without shaking, MIC of VAN to inhibit MRSA growth was measured by a Biotek Synergy H1 hybrid multi-mode microplate reader (BioTek, Winooski, Vt., USA) at OD₆₀₀. To evaluate the killing time of VAN to bacteria in stationary phase (1×10⁸ CFU mL⁻¹), VAN powder was added to bacteria solution to obtain concentration of 1 mg mL⁻¹, 3 mg mL⁻¹, 5 mg mL⁻¹, and 10 mg mL⁻¹, respectively, while remaining MRSA concentration of 1×10⁸ CFU mL⁻¹. After incubation in a 37° C. water bath for 1 day, 2 days, 3 days and 4 days, the CFU of MRSA cells was tested by the standard plate spread counting assay, respectively. The time-killing tests of DAP with different concentrations (0.1 mg mL⁻¹, 0.5 mg mL⁻¹, 1 mg mL⁻¹, and 5 mg mL⁻¹) on MRSA planktonic cells were also performed, with the incubation time up to 24 hours.

### Combined Biofilm Treatment Efficacy of Electrical Debridement and High-Concentration Antibiotics Delivery

The HIC-based biofilm treatment system 100 embodiment shown in FIG. 18 was used to evaluate combined biofilm treatment efficacy of electrical debridement and VAN delivery. To evaluate the anti-biofilm treatment efficacy of current intensity, 0 mA cm⁻² (passive diffusion) to 75 mA cm⁻² was applied on the HIC-based biofilm treatment system 100 with 1 mg mL⁻¹ VAN loaded in HIC-based working device 104, respectively. To evaluate the anti-biofilm treatment efficacy of loading concentration of VAN, 1 mg mL⁻¹, 5 mg mL⁻¹, and 10 mg mL⁻¹ VAN was loaded in the drug/buffer chamber 114 of the HIC-based working device 104, respectively, and applied 75 mA cm⁻². To further enhance the anti-biofilm efficacy of electrical debridement and high concentration delivery of VAN, two treatments were applied, named protocol #1 and protocol #2. Protocol #1 applied two treatments (treatment: 75 mA cm⁻² for 1 hour, 1 mg mL⁻¹ VAN loaded in HIC-based working device 104) separated by 6 hours. Protocol #2 applied two treatments (treatment same as Protocol #1) separated by 24 hours. CFU was counted 24 hours after last treatment using the standard spread counting assay. The bactericidal efficacy was calculated accordingly.

The combined biofilm treatment efficacy of electrical debridement and DAP delivery was also evaluated. The test setup was same as that of VAN, except no PBS chamber 122 was used. Instead, the inner surface of the skin wound contacted directly to the hydrogel 120 of the counter device 106. The 0 mA cm⁻² (passive diffusion) to 75 mA cm⁻² were applied on the test system with 5 mg mL⁻¹ DAP loaded in HIC-based working device 104 to measure the effect of current intensity on the anti-biofilm efficacy. 1 mg mL⁻¹, 5 mg mL⁻¹, and 10 mg mL⁻¹ DAP was loaded in HIC-based working device 104, respectively, and applied 75 mA cm⁻² to evaluate the anti-biofilm treatment efficacy of DAP loading concentration. Calcium chloride solution (1 mL, 100 mg mL⁻¹) was supplied on the infected wound and allowed for 24-hour passive diffusion after DAP treatment, since DAP require calcium ion to have anti-bacteria function. CFU was counted immediately after calcium ions 24-hour diffusion using the standard spread counting assay. A protocol was developed with two steps to reduce the treatment time. For step 1, biofilm-infected skin wound was treated with 75 mA cm⁻² for 1 hour induced by HIC-based cathode device, loaded with 5 mg mL⁻¹ DAP. Immediately after step 1, HIC-based anode device was applied on the wound loaded with calcium chloride solution (100 mg mL⁻¹) and applied 75 mA cm⁻² for 5 minutes (step 2). CFU was counted 24 hours after the treatment of step 2. The bactericidal efficacy was calculated accordingly.

To evaluate the paradoxical effect of VAN, the ex vivo mature MRSA biofilm established on the porcine skin wound was incubated in 1 mg mL⁻¹, 4.5 mg mL⁻¹, 10 mg mL⁻¹, and 20 mg mL⁻¹ VAN for passive diffusion of VAN, respectively. After 24 hours incubation, the CFU of biofilm infected wounds under different VAN treating concentrations were measured.

### In Vivo Safety Test of High-Intensity Current Application

BALB/c mouse (6-10 weeks, ˜20 g) were purchased from the Jackson Laboratory (Bar Harbor, Me., USA). The animal test was incompliance with the protocol approved by the Institutional Animal Care and Use Committee (IACUC) at the University of Nebraska Medical Center (protocol #21-049-06-FC). To evaluate the safety of high current intensity on the mouse skin, the drug/buffer chamber 114 (diameter: 4 mm) of anode HIC-based working device 104 was mounted on the back of mouse with the help of 3M Tegaderm transparent film after mouse were anesthetized and after hair removal. After adding 0.5 mL PBS in the drug/buffer chamber 114, the HIC-based hydrogel 112 and phosphate salt solution chamber 110 were attached on the drug/buffer chamber 114. Different current intensities (0 mA cm⁻² (sham control), 19 mA cm², 38 mA cm⁻², and 75 mA cm⁻²) were applied from a DC power supply 102 and route to the mouse skin. The counter device 106 was also attached to the back of mouse skin to complete the circuit. The HIC-based counter device 106 had larger skin contact area (diameter: 7 mm) to minimize the pH/temperature impact of the counter device 106. A conventional electrical device (see FIG. 2B) with the carbon electrode inserted in drug/buffer chamber directly was used as a comparison. 8 mA cm⁻² was induced by the conventional device and lasted for 1 hour. The pH of skin surface immediately after test was measured by using a flat pH probe. Digital images of the skin were taken to inspect for signs of redness, blisters, and tissue damage 4 hours and 24 hours after different treatments. At 24 hour post treatment, each mouse was sacrificed, and the skin tissues were collected for histological evaluation (Hematoxylin and Eosin (H&E) staining, Masson's trichrome stain, and Verhoeff-Van Gieson (VVG) staining).

In vivo Anti-Biofilm Efficacy Test of the HIC-Based Biofilm Treatment System

The in vivo anti-biofilm efficacy test was incompliance with the protocol approved by the Institutional Animal Care and Use Committee of the University of Nebraska Medical Center (protocol #21-049-06-FC). MRSA was cultured with LB media in water shaking bath (37° C.) overnight. Then 100 μL bacteria was transferred to 4 mL LB media and cultured for another 4 hours to reach the density of 1×10⁸ mL⁻¹ and stored in ice before use. In this study, 000697-B6.BKS(D)-Leprdb/J mouse (5-6 weeks, around 30 g) was used to establish the biofilm infected skin wound model. Specifically, after mouse anesthesia and hair removal, one full-thickness wound (4 mm in diameter) was created on the back of the mouse by using 4-mm biopsy punch. For negative control group, two wounds were created on the back of the mouse. Bacteria solution (10 μL) was carefully dropped in the wound site. Then the infected wound was covered and sealed by 3M Tegaderm transparent film. After mouse was infected for 2 days, different anti-biofilm treatments were applied on the wound, respectively. Working device 104 was attached on the wound site with the help of 3M Tegaderm transparent film. Cathode phosphate salt solution (2 mL) was loaded in phosphate salt solution chamber 122 and PBS/daptomycin solution (0.5 mL) was added in the drug/buffer chamber 114. The effective treatment area was 0.125 cm² to fully cover the infected wound site. The counter device 106 attached to the belly of the mouse directly down to the working device 104 to complete the circuit. To evaluate the anti-biofilm efficacy of high-intensity current alone, 75 mA cm⁻² ionic current was applied in the system for 60 minutes with PBS loaded in the drug/buffer chamber 114. To evaluate the anti-biofilm efficacy of low-intensity current combined with DAP, 0.5 mA cm⁻² ionic current was applied in the system for 60 minutes with DAP (5 mg mL⁻¹) loaded in the drug/buffer chamber 114. To evaluate the efficacy of the H IC-based biofilm treatment system, 75 mA cm⁻² ionic current was applied in the system for 60 minutes with DAP (5 mg mL⁻¹) loaded in the drug/buffer chamber 114. To perform the antibacterial function of DAP, immediately after the treatment of each group, HIC-anode device was applied on the wound loaded with calcium chloride solution (100 mg mL⁻¹) and applied 75 mA cm⁻² for 5 minutes. The infected wound without treatment was used as the negative control. Then, 24 hours or 7 days after treatment, the infected skin wounds and surrounding tissues were collected by 6-mm biopsy punch. Bacterial count per gram was evaluated using the standard plate counting assay.

### Statistical Analysis

The statistical difference between samples were determined by unpaired student's t-tests (GraphPad Software, SanDiego, Calif., USA). The statistical difference in the figures were present by * for p<0.05, ** for p<0.01, *** for p<0.005, and **** for p<0.001. At least 3 replicates were performed for all statistics.

Further Application of the HIC-Based Biofilm Treatment System to Inhibit the Formation, Reformation, and/or Growth of Biofilm

Biofilm infection can lead to chronic, non-healing wounds (CWs). The current clinical care for CW biofilm infection uses debridement and antibiotic treatment. Debridement removes biofilm-contaminated tissue and reduces bacterial load. However, biofilm can quickly recover after debridement, so repeated debridement is required. Antibiotics administrated via systemic or topical routes have a low bioavailability in wound tissue, necessitating long-term or high-frequency dosing to maintain a low bacterial count in CWs. The current treatment imposes a heavy burden on patients and on the healthcare system.

Nanoparticle-based antibiotic formulations allow sustained drug release and can potentially reduce antibiotic dosing frequency. However, topical application, the most common route of nanoparticle administration, has a low delivery efficiency due to the slow diffusion process, leading to unsatisfactory biofilm inhibition. There is a critical need for a high-efficiency nanoparticle delivery technology that can rapidly deliver a therapeutic dose of antibiotic nanoparticles into CWs to achieve a long-term inhibition of biofilm infection.

The HIC-based biofilm treatment system 100 was tested for the delivery of vancomycin-encapsulated nanoparticles (PLGA/Van) to inhibit the formation, reformation, and/or growth of biofilm. The HIC-based biofilm treatment system 100 has previously shown enhanced safety for high-intensity current applications and increased delivery efficiency of free antibiotics into biofilm-infected wound tissue. Here, it was further demonstrated that the HIC-based biofilm treatment system 100 was able to significantly increase the delivery efficiency of PLGA/Van into ex vivo porcine skin wounds compared to conventional low-intensity iontophoresis and diffusion. The higher amount of PLGA/Van delivered by HIC-based biofilm treatment system 100 achieved a better biofilm inhibition efficacy.

In experiments, PLGA/Van was synthesized using double-emulsion methods. 40 mg Resomer-RG653H was dissolved in 2 mL dichloromethane and emulsified with 0.5 mL 1% vancomycin solution on ice using probe ultrasonicator. Primary emulsion was then mixed with 10 mL solution containing 1% PVA and 0.01% chitosan and emulsified again. Secondary emulsion was subsequently stirred for 6 hours.

Nanoparticles were isolated and washed in 50 kDa cut-off centrifugal filters. For unloaded Poly (lactic-co-glycolic acid) (PLGA) nanoparticles, 0.5 mL H₂O was used instead in primary emulsion.

Vancomycin release was monitored using Slide-A-Lyzer™ Dialysis Devices. 5 mg PLGA/Van was dialyzed against MilliQ-water at 37° C., from which samples were withdrawn periodically for UV-Vis measurements. Same volume of MilliQ-water was supplemented afterwards.

PEG hydrogel (10% PEGDMA MW8000, 5% PEGDA MW700, and 1% Irgacure-2959) was fabricated with UV crosslinking and was attached to the device using benzophenone-assisted bonding. The anode solution contains 0.6M Na₂HPO₄, and the cathode solution contains 0.6M NaH₂PO₄ and 0.48M Na₂HPO₄. Carbon rods were used to supply electric current to the HIC-based biofilm treatment system 100.

Iontophoretic nanoparticle delivery was conducted on 6-mm excisional porcine skin wounds and quantified via UV-Vis measurements after extracting the treated biopsy in 2% acetic acid and acetonitrile. Delivered nanoparticles were allowed to release vancomycin for 3 days before wounds were inoculated with Methicillin-resistant Staphylococcus aureus (MRSA) at 10⁸ CFU/mL. Wounds were then incubated at 37° C. for 2 days before bacterial quantification.

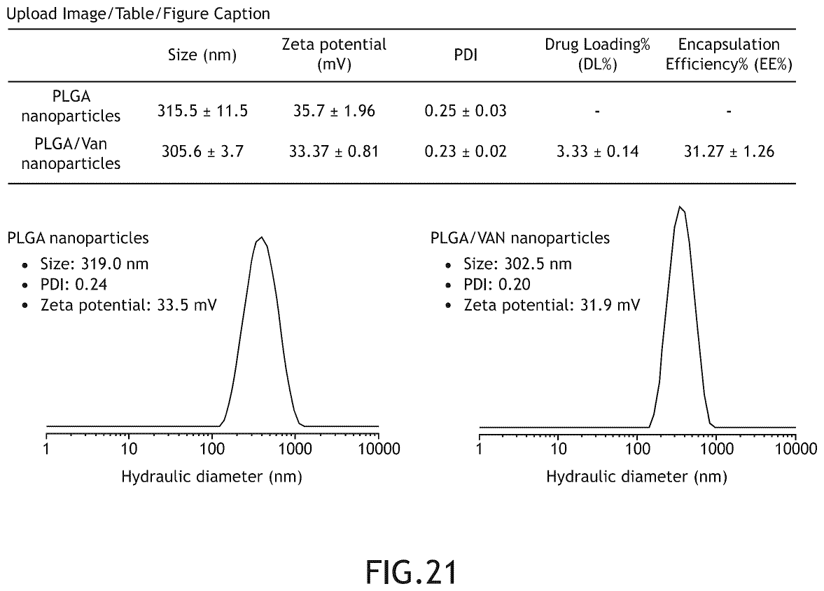

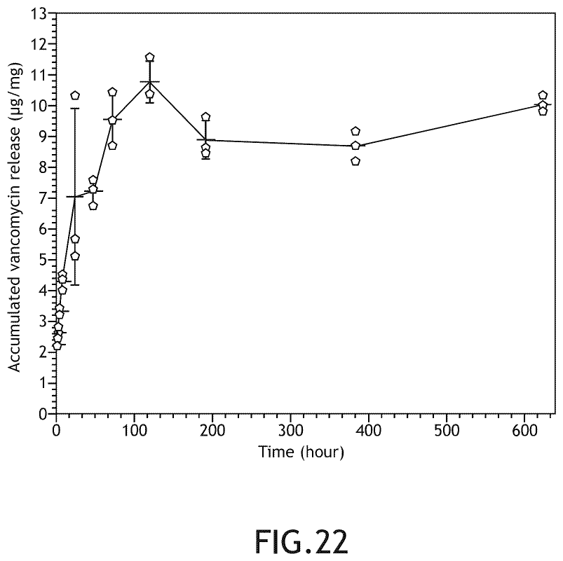

Both PLGA/Van and unloaded PLGA nanoparticles had an average size of ˜300 nm, were monodispersed (PDI<0.3) and positively charged (zeta potential >30mV). DL % and EE % (see FIG. 21) were consistent with what others have reported. Vancomycin release was continuously monitored for 26 days. The results demonstrated that vancomycin released fast in the first 3 days. In total 9.5 μg vancomycin per mg of PLGA/Van was released by Day 3 (see FIG. 22). This increased to 10.8 μg/mg on Day 5, which accounted for 32.7% of vancomycin loaded in PLGA/Van. The release did not increase significantly from Day 5 to 26.

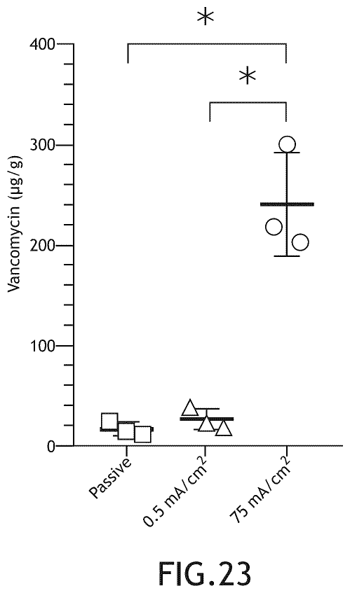

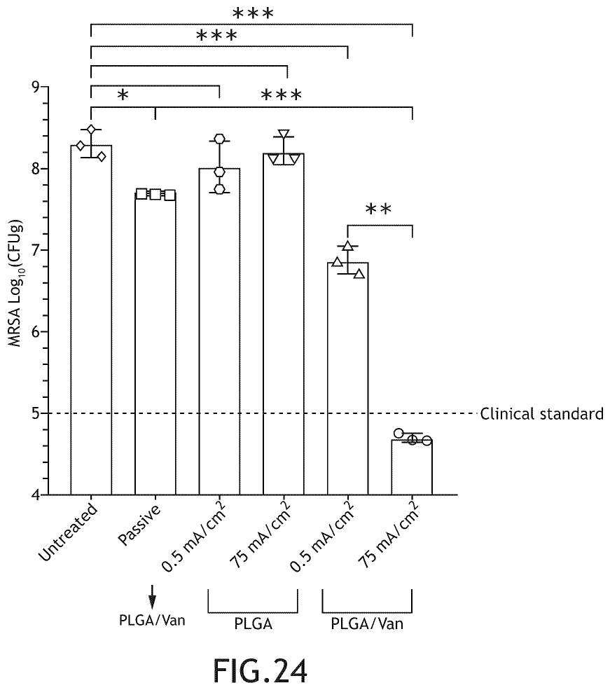

PLGA/Van delivery was performed using HIC-based biofilm treatment system 100. 20 mg PLGA/Van dispersed in PBS was loaded in drug/buffer chamber 114. A high current density of 75 mA/cm² was used to deliver PLGA/Van into ex vivo porcine skin wound for 1 hour. A conventional low-intensity iontophoresis at 0.5 mA/cm² and passive diffusion were also tested as comparisons. 240.2 μg/g vancomycin (7.21 mg/g PLGA/Van) was delivered into wound tissue using 75 mA/cm² iontophoresis, while only 26.2 and 16.3 μg/g vancomycin (0.79 and 0.49 mg/g PLGA/Van) were delivered by 0.5 mA/cm 2 iontophoresis and passive diffusion (see FIG. 23). Treated wounds were further challenged with MRSA at 3-day post-delivery to evaluate biofilm inhibition efficacy. 10^{8.30} CFU/g biofilm was harvested from untreated wounds at Day 2 post-inoculation, similar to those treated with unloaded PLGA nanoparticles (10^{8.02} and 10^{8.21} CFU/g) (see FIG. 24). PLGA/Van delivered by passive diffusion and 0.5 mA/cm² iontophoresis achieved low bacterial reductions of 0.62 and 1.43 Log10-scales respectively, compared with untreated wounds. However, PLGA/Van delivered by 75 mA/cm² iontophoresis achieved a significantly higher biofilm inhibition efficacy. The bacterial count was measured to be 10^{4.70} CFU/g on Day 3 (3.61 Log10-scales lower than untreated control), which was below clinical threshold for wound infections.

From the results provided above, it was concluded that using the HIC-based biofilm treatment system 100 significantly enhanced antibiotic nanoparticle delivery efficiency into ex vivo porcine skin wounds and achieved better biofilm inhibition efficacy. It is contemplated that the HIC-based biofilm treatment system 100 will also enhance biofilm inhibition efficacy in an in vivo wound infection model and improve the wound healing process.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

What is claimed is:

1. A hydrogel ionic circuit (HIC)-based electrical biofilm treatment system, comprising:
a working device that includes:
a first chamber containing a salt solution;
a second chamber containing a therapeutic solution, the second chamber being configured to interface with a surface overlaying a target region;
a hydrogel membrane separating the first chamber from the second chamber; and
an electrode configured to apply an electrical current to the first chamber of the working device to induce an ion current in the salt solution, wherein the ion current acts on the second chamber to iontophoretically transport molecules from the therapeutic solution across the surface to the target region; and
a counter device that includes:
a third chamber containing a same or different salt solution;
a fourth chamber containing a buffer solution, the fourth chamber being configured to interface with a second surface on an opposite side of or near the surface overlaying the target region;
a second hydrogel membrane separating the third chamber from the fourth chamber; and
a counter electrode connected to the third chamber of the counter device.

2. The HIC-based electrical biofilm treatment system of claim 1, wherein the hydrogel membrane is ionically conductive and configured to transmit the ion current to the second chamber.

3. The HIC-based electrical biofilm treatment system of claim 1, wherein the hydrogel membrane comprises a polyethylene glycol (PEG) hydrogel matrix.

4. The HIC-based electrical biofilm treatment system of claim 1, wherein the hydrogel membrane is configured to contain salt ions stably within the first chamber due to aqueous two-phase separation (ATPS).

5. The HIC-based electrical biofilm treatment system of claim 1, wherein the salt solution comprises at least one of: a sodium chloride solution, a sodium phosphate solution, a potassium chloride solution, a sodium dihydrogen phosphate solution, or a disodium phosphate solution.

6. The HIC-based electrical biofilm treatment system of claim 1, wherein the salt solution contains an amount of salt required to maintain a pH in the range of 6.5 to 8.5 at the surface overlaying the target region.

7. The HIC-based electrical biofilm treatment system of claim 1, wherein the salt solution is configured to absorb heat generated by electrode overpotential to maintain a temperature below 43° C. at the surface overlaying the target region.

8. The HIC-based electrical biofilm treatment system of claim 1, wherein the target region is a cutaneous wound.

9. The HIC-based electrical biofilm treatment system of claim 1, wherein the therapeutic solution includes an antibiotic and/or antimicrobial agent.

10. The HIC-based electrical biofilm treatment system of claim 9, wherein the antibiotic and/or antimicrobial agent comprises vancomycin or daptomycin.

11. The HIC-based electrical biofilm treatment system of claim 1, wherein the working device is configured to debride biofilm at the surface overlaying the target region with electrostatic force generated by the ion current acting on the second chamber.

12. A hydrogel ionic circuit (HIC)-based device for therapeutic iontophoresis and/or biofilm debridement, comprising:
a first chamber containing a salt solution;
a second chamber containing a therapeutic or buffer solution, the second chamber being configured to interface with a surface overlaying a target region;
a hydrogel membrane separating the first chamber from the second chamber; and
an electrode configured to apply an electrical current to the first chamber of the working device to induce an ion current in the salt solution, wherein the ion current acts on the second chamber to iontophoretically transport therapeutic molecules across the surface overlaying the target region and/or debride biofilm at the surface overlaying the target region.

13. The HIC-based device of claim 12, wherein the hydrogel membrane is ionically conductive and configured to transmit the ion current to the second chamber.

14. The HIC-based device of claim 12, wherein the hydrogel membrane comprises a polyethylene glycol (PEG) hydrogel matrix.

15. The HIC-based device of claim 12, wherein the hydrogel membrane is configured to contain salt ions stably within the first chamber due to aqueous two-phase separation (ATPS).

16. The HIC-based device of claim 12, wherein the salt solution comprises at least one of: a sodium chloride solution, a sodium phosphate solution, a potassium chloride solution, a sodium dihydrogen phosphate solution, or a disodium phosphate solution.

17. The HIC-based device of claim 12, wherein the salt solution contains an amount of salt required to maintain a pH in the range of 6.5 to 8.5 at the surface overlaying the target region, and wherein the salt solution is configured to absorb heat generated by electrode overpotential to maintain a temperature below 43° C. at the surface overlaying the target region.

18. The HIC-based device of claim 12, wherein the target region is a cutaneous wound.

19. A method of treating infectious biofilm affecting a wound surface, the method comprising:
disposing a salt solution within a first chamber;
disposing a therapeutic or buffer solution within a second chamber, wherein the first chamber and the second chamber are separated by a hydrogel membrane;
interfacing the second chamber with the wound surface; and
applying an electrical current to the first chamber to induce an ion current in the salt solution, wherein the ion current acts on the second chamber to iontophoretically transport therapeutic molecules across the wound surface and/or debride biofilm at the wound surface.

20. A method of treating a wound surface to inhibit the formation, reformation, and/or growth of biofilm, the method comprising:
disposing a salt solution within a first chamber;
disposing an antibiotic and/or antimicrobial solution within a second chamber, wherein the first chamber and the second chamber are separated by a hydrogel membrane;
interfacing the second chamber with the wound surface; and
applying an electrical current to the first chamber to induce an ion current in the salt solution, wherein the ion current acts on the second chamber to iontophoretically transport antibiotic and/or antimicrobial molecules across the wound surface.
